(19) United States
(12) Patent Application Publication
(10) Pub. No.: US 2018/0303802 A1
(43) Pub. Date: Oct. 25, 2018

(54) METHODS FOR TREATING SYNOVIAL SARCOMA

(71) Applicant: THE BOARD OF TRUSTEES OF THE LELAND STANFORD JUNIOR UNIVERSITY, Stanford, CA (US)

(72) Inventors: Cigall KADOCH, Tiburon, CA (US); Gerald R. CRABTREE, Woodside, CA (US)

(21) Appl. No.: 15/766,192

(22) PCT Filed: Oct. 5, 2016
(86) PCT No.: PCT/US2016/055578

§ 371 Date: Apr. 5, 2018

Related U.S. Application Data
(60) Provisional application No. 62/237,369, filed on Oct. 5, 2015.

(51) Int. Cl. A61K 31/427; A61P 35/00; A61K 31/395; A61K 31/437

(52) U.S. Cl. CPC A61K 31/427; A61K 31/437; A61K 31/395; A61P 35/00

(57) ABSTRACT

Methods useful in the treatment of synovial sarcoma are provided. The methods comprise administering to a subject suffering from synovial sarcoma a compound as disclosed herein. Also provided are novel compounds having therapeutic effects on subjects suffering from synovial sarcoma and pharmaceutical compositions comprising the compounds. The compounds were identified by screening for agents that promote the assembly of wild-type BAF (also called mSWI/SNF) complexes in modified SS cells.

### CROSS-REFERENCE TO RELATED APPLICATION

This application claims the benefit of U.S. Provisional Application No. 62/237,369, filed on Oct. 5, 2015, the disclosure of which is incorporated herein by reference in its entirety.

### STATEMENT OF GOVERNMENTAL SUPPORT

This invention was made with government support under grant number RO1NS046789 awarded by the National Institutes of Neurological Disorders and Stroke and grant number RO1CA163915 awarded by the National Cancer Institute. The government has certain rights in this invention.

### BACKGROUND OF THE INVENTION

Chromatin regulation, for example by DNA methylation, histone modification, or ATP-dependent chromatin remodeling, is essential for appropriate and timely gene expression. The SWI/SNF (BAF) complex is one of the best characterized chromatin remodeling complexes. It plays a role in gene activation through the remodeling of nucleosomes, thus allowing transcription factors access to their recognition sites.

Mutations to subunits of polymorphic BAF complexes have repeatedly been identified by exome sequencing of primary, early cancers. Indeed, recent exon-sequencing studies of human tumors have revealed that subunits of BAF are mutated in more than 20% of all human malignancies (Kadoch et al. (2013) Nature Genet. 45:592; You et al. (2012) Cancer Cell 22:9), but the mechanisms involved in tumour suppression are unclear. BAF chromatin-remodelling complexes are polymorphic assemblies that use energy provided by ATP hydrolysis to regulate transcription through the control of chromatin structure (Clapier et al. (2009) Annu. Rev. Biochem. 78:273)) and the placement of Polycomb Repressive Complex 2 (PRC2) across the genome (Ho et al. (2011) Nature Cell Biol. 13:903; Wilson et al. (2010) Cancer Cell 18:316). Several proteins dedicated to this multisubunit complex, including BRG1 [SMARCA4] and BAF250a [ARID1A], are mutated at frequencies similar to those of recognized tumour suppressors. In particular, the core ATPase BRG1 is mutated in 5-10% of childhood medulloblastomas (Parsons et al. (2011) Science 331:435; Pugh et al. (2012) Nature 488:106; Jones et al. (2012) Nature 488:100; Robinson et al. (2012) Nature 488:43) and more than 15% of Burkitt's lymphomas (Love et al. (2012) Nature Genet. 44:1321; Richter et al. (2012) Nature Genet. 44:1316).

A recent study demonstrated a previously unknown function of BAF complexes in decatenating newly replicated sister chromatids, a requirement for proper chromosome segregation during mitosis. Dykhuizen et al. (2013) Nature 497:624. These results have been used to develop methods for identifying and treating cancer patients likely to respond to topoisomerase inhibitors or likely to fail to respond to topoisomerase inhibitors. See US Patent Application No. 2015/0185221A1.

Human synovial sarcoma (SS) is a soft tissue sarcoma that is associated with a translocation event, t(X;18)(p11.2;q11.2), which fuses the SS18 gene on chromosome 18 to one of three closely related genes-SSX1, SSX2, or SSX4-on the X chromosome, resulting in an in-frame fusion protein in which the eight C-terminal amino acids of SS18 are replaced with 78 amino acids from the SSX C-terminus. Kadoch et al. (2013) Cell 153:71. This type of sarcoma accounts for about 8-10% of all soft-tissue malignancies and commonly occurs in the extremities of young adults and pediatric patients at inaccessible locations, which are often discovered late in the course of the disease. The malignancies are generally refractory to conventional chemotherapy-based forms of treatment; except for a small percentage of cases in which the tumors can be successfully removed with surgery, they are nearly always lethal. Methods and compositions for treating human synovial sarcoma, as well as screens to identify therapeutics for such treatment, have been reported. US Patent Application No. 2014/0288162A1. Specific therapeutic agents have, however, not yet been identified.

The above underscores a significant need for novel approaches in the treatment of synovial sarcoma and related diseases.

### SUMMARY OF THE INVENTION

The present invention addresses these and other problems by providing, in one aspect, novel methods for treating synovial sarcoma.

In particular, according to this aspect of the invention, methods of treatment are provided that comprise administering to a subject in need thereof a concentration of a compound sufficient to treat the subject, wherein the compound is represented by structural formula (I):

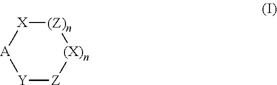

or a stereoisomer, tautomer, or pharmaceutically acceptable salt thereof, wherein:
A is an optionally substituted bivalent aryl or heteroaryl group;
each X is independently O or S;
Y is —C(O)NR₁— or —C(S)NR₁—, wherein R₁ is hydrogen, alkyl, alkenyl, alkynyl, aryl, aralkyl, heteroaryl, heteroaralkyl, cycloalkyl, cycloalkenyl, heterocyclyl, or heterocyclylalky, and is optionally substituted with alkyl, alkenyl, alkynyl, alkoxy, alkanoyl, alkylamino, alkylthio, aryl, aryloxy, arylamino, aralkyl, aralkoxy, aralkanoyl, aralkamino, heteroaryl, heteroaryloxy, heteroarylamino, heteroaralkyl, heteroaralkoxy, heteroaralkanoyl, heteroaralkamino, cycloalkyl, cycloalkenyl, cycloalkoxy, cycloalkanoyl, cycloalkamino, heterocyclyl, heterocyclyloxy, heterocyclylamino, heterocyclylalky, heterocyclylalkoxy, heterocyclylalkanoyl, heterocyclylalkamino, hydroxyl, thio, amino, amido, alkanoylamino, aroylamino, aralkanoylamino, alkylcarboxy, alkylcarbonyl, aminocarbonyl, alkylaminocarbonyl, carboxy, carbonate, carbamate, guanidinyl, urea, halo, trihalomethyl, cyano, nitro, phosphoryl, sulfonyl, sulfonamido, or azido;
each Z is independently an optionally substituted C₂₋₆ alkylene group; and
each n is independently either 0 or 1.

Also provided according to this aspect of the invention are methods of treatment that comprise administering to a subject in need thereof a concentration of a compound sufficient to treat the subject, wherein the compound is represented by structural formula (II):

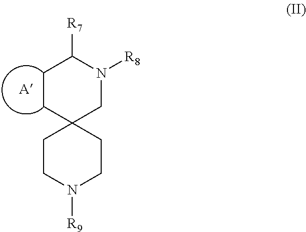

or a stereoisomer, tautomer, or pharmaceutically acceptable salt thereof, wherein:
A′ is an optionally substituted aryl or heteroaryl ring system; and
R₇, R₈, and R₉ are each independently, alkyl, alkenyl, alkynyl, acyl, aryl, aralkyl, heteroaryl, heteroaralkyl, cycloalkyl, cycloalkenyl, heterocyclyl, or heterocyclylalky, and is optionally substituted with alkyl, alkenyl, alkynyl, alkoxy, alkanoyl, alkylamino, alkylthio, aryl, aryloxy, arylamino, aralkyl, aralkoxy, aralkanoyl, aralkamino, heteroaryl, heteroaryloxy, heteroarylamino, heteroaralkyl, heteroaralkoxy, heteroaralkanoyl, heteroaralkamino, cycloalkyl, cycloalkenyl, cycloalkoxy, cycloalkanoyl, cycloalkamino, heterocyclyl, heterocyclyloxy, heterocyclylamino, heterocyclylalky, heterocyclylalkoxy, heterocyclylalkanoyl, heterocyclylalkamino, hydroxyl, thio, amino, amido, alkanoylamino, aroylamino, aralkanoylamino, alkylcarboxy, alkylcarbonyl, aminocarbonyl, alkylaminocarbonyl, carboxy, carbonate, carbamate, guanidinyl, urea, halo, trihalomethyl, cyano, nitro, phosphoryl, sulfonyl, sulfonamido, or azido.

### BRIEF DESCRIPTION OF THE DRAWINGS

FIGS. 1A-1B illustrate the development of a luciferase-tagged BAF47 fusion protein for use in screening small-molecule libraries. FIG. 1A: Immunoprecipitation of BAF complexes in WT (293T, B35) versus SS (Aska, Yamato) cells modified with BAF47-Luc. FIG. 1B: BAF47-Luc modified Aska-SS cells exhibit induction of BAF47-Luc levels upon complex reversion using either shSS18-SSX1 or overexpression of WT SS18.

FIGS. 2A-2B show the results of high-throughput screening of a Diversity Oriented Synthesis (DOS) small-molecule library using the screening methods developed and optimized herein. FIG. 2A: Average compound activity plot showing hits (highest replicative normalized activities), inconclusives (non-replicative normalized activities), and inactives (lowest normalized activities). Actives scored at >45% activity (3×SD of mean), with values normalized to the DMSO treated population (no positive control). FIG. 2B: Replicate plot reveals 33 positive hits with >45% activity (luminescence value); 0.03% hit rate from total library of 97,489 DOS compounds screened in 4 parallel high-throughput screening (HTS) runs. 591 inconclusives were scored (0.6%), showing signal in only one of two replicates. Cell/assay performance was validated by repeating validation plates at the end of the final run.

FIGS. 3A-3B show the validation of compound hits in unmodified Aska-SS cells using compounds identified in the HTS assays. FIG. 3A: Immunoprecipitations not normalized for total protein input. FIG. 3B: Immunoprecipitations normalized for a total protein input of 70 μg.

### DETAILED DESCRIPTION OF THE INVENTION

The instant application discloses the design, optimization, and use of highly efficient screening assays to identify compounds effective in the disruption of oncogenic BAF complex formation from small-molecule libraries. The application further discloses methods of treatment comprising administering to a subject in need thereof compounds identified in the above screening assays. The screening assays make use of a type of cancer, human synovial sarcoma, where nearly all tumors have a precise translocation involving a specific subunit. This feature indicates that the translocation is the initiating oncogenic event. The compounds identified in the assays are thus effective in the treatment of synovial sarcoma.

In particular, the instant inventors have previously demonstrated that SS18 is a dedicated, highly stable subunit of BAF complexes. See Kadoch et al. (2013) Cell 153:71, which is incorporated by reference herein in its entirety. According to those findings, the fusion of SS18 with SSX produces a protein that binds to the complex and evicts both the wild-type SS18 and the tumor suppressor BAF47. The altered complex then binds to Sox2, relieving H3K27me3 repression, and thereby activating Sox2 and thus cell proliferation. Furthermore, the disruption of the BAF complex, as driven by the SS18-SSX fusion protein, is determined by a 2 amino acid hydrophilic region of SSX.

Screening assays were previously described by the instant inventors in US Patent Application Publication No. 2014/0288162A1, which is incorporated by reference herein in its entirety. In those screens, a gain-of-function method to detect molecules with the ability to favor the assembly of the normal BAF complex was described. The screen relied on the discovery that incorporation of the SS18-SSX fusion protein leads to eviction of BAF47 and its subsequent destabilization and proteasome-mediated degradation. Hence, a molecule that favors assembly of normal BAF complexes leads to increased levels of the BAF47 protein by virtue of its ability to assembly into complexes and its subsequent stabilization (as demonstrated with either shRNA-mediated knock down of the SS18-SSX fusion or by overexpression of wild-type SS18).

A lentiviral delivery construct was described that tagged firefly luciferase to the C-terminus of full length BAF47. Introduction of tagged BAF47 into two SS cell lines results in minimally detectable total protein levels, as well as BAF47 protein levels on BAF complexes, as assessed by anti-Brg immunoprecipitation studies. Upon co-introduction of wild-type SS18 or upon knockdown of the SS18-SSX fusion protein, increased BAF47 total protein levels and BAF-associated protein is observed. Therefore, small molecules that lead to the re-assembly of BAF47 into complexes will lead to an increase in luciferase signal, (i.e. a gain-of-function). This gain-of-function approach has the advantage in that it will eliminate non-specific toxic molecules that simply kill the cell or impair transcription, translation or protein stability.

As previously disclosed, in the synovial sarcoma cell lines Aska-SS and Yamato-SS, BAF47-luciferase is evicted from BAF complexes leading to its destabilization. In the presence of a small molecule, e.g. a small molecule that binds to the K43-R44 amino acids of SSX1, SSX2 or SSX4, it is expected that the transforming SS18-SSX fusion will not be able to assemble into BAF complexes. Without intending to be bound by theory, it is further expected that this class of synovial sarcoma therapeutics will have the general features of one hydrophobic side (which will mimic the M, I residues in the non-transforming SSX family members) and one hydrophilic side that will bind the hydrophobic K43, R44 residues. In the presence of such small molecules BAF47 will be incorporated into complexes, leading to its stabilization (as detected by increased luciferase signal), reduced Sox2 expression, and cessation of proliferation. Compounds identified in the screens are thus useful in the treatment of cancers such as synovial sarcoma.

### Methods of Treatment

Accordingly, in one aspect, the invention provides novel methods of treatment that comprise administering to a subject in need thereof a concentration of a compound sufficient to treat synovial sarcoma in the subject. The subject compounds may be administered by any route suitable for achieving the desired effect. For example, the therapeutic compound may be administered orally, intravenously, inhalationally, subcutaneously, intramuscularly, transdermally, topically, or by any other suitable route.

By a “synovial sarcoma” it is meant a soft tissue sarcoma that is associated with the translocation event t(X;18)(p11.2;q11.2), which fuses the coding sequence for the first 379 amino acids of the SS18 gene on chromosome 18 to the coding sequence for the last 78 amino acids one of three closely related genes-SSX1, SSX2, or SSX4-on the X chromosome. In other words, the C-terminal 78 amino acids of SSX1, SSX2, or SSX4 become fused to SS18 at residue 379. The term is understood to include tumor cells containing any of the above genetic translocation events and/or expressing any of the above fusion proteins.

Individuals having a synovial sarcoma may be readily identified in any of a number of ways. For example, a cytogenetics assay, e.g. a chromosomal analysis, e.g. chromosomal smear, may be used in diagnosing a synovial sarcoma. As a second example, although synovial sarcomas have been documented in most human tissues and organs, including brain, prostate, and heart, synovial sarcomas have a propensity to arise adjacent to joints, e.g. large joints of the arm and leg. As such, the detection of a sarcoma in a joint, e.g. a large joint of the arm or leg, may be used in diagnosing a synovial sarcoma. As a third example, synovial sarcomas comprise 2 types of cells. The first type, known as a spindle or sarcomatous cell, is relatively small and uniform, and found in sheets. The other is epithelial in appearance. Classical synovial sarcoma has a biphasic appearance with both types present. Synovial sarcoma can also appear to be poorly differentiated or to be monophasic fibrous, consisting only of sheets of spindle cells. As such, a histological analysis of an SS biopsy may be used in diagnosing a synovial sarcoma.

In some embodiments, the compound used in the instant methods of treatment is represented by structural formula (I):

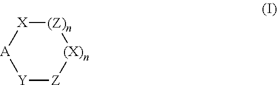

or a stereoisomer, tautomer, or pharmaceutically acceptable salt thereof;
wherein A is an optionally substituted bivalent aryl or heteroaryl group;
each X is independently O or S;
Y is —C(O)NR₁— or —C(S)NR₁—, wherein R₁ is hydrogen, alkyl, alkenyl, alkynyl, aryl, aralkyl, heteroaryl, heteroaralkyl, cycloalkyl, cycloalkenyl, heterocyclyl, or heterocyclylalky, and is optionally substituted with alkyl, alkenyl, alkynyl, alkoxy, alkanoyl, alkylamino, alkylthio, aryl, aryloxy, arylamino, aralkyl, aralkoxy, aralkanoyl, aralkamino, heteroaryl, heteroaryloxy, heteroarylamino, heteroaralkyl, heteroaralkoxy, heteroaralkanoyl, heteroaralkamino, cycloalkyl, cycloalkenyl, cycloalkoxy, cycloalkanoyl, cycloalkamino, heterocyclyl, heterocyclyloxy, heterocyclylamino, heterocyclylalky, heterocyclylalkoxy, heterocyclylalkanoyl, heterocyclylalkamino, hydroxyl, thio, amino, amido, alkanoylamino, aroylamino, aralkanoylamino, alkylcarboxy, alkylcarbonyl, aminocarbonyl, alkylaminocarbonyl, carboxy, carbonate, carbamate, guanidinyl, urea, halo, trihalomethyl, cyano, nitro, phosphoryl, sulfonyl, sulfonamido, or azido;
each Z is independently an optionally substituted C₂₋₆ alkylene group; and
each n is independently either 0 or 1.

In more specific embodiments, the compound is represented by structural formula (I);
wherein A is

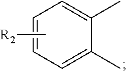

R₂ is hydrogen, —N(R₃)₂, —NR₃C(O)R₄, or —NR₃C(O)N(R₄)₂;
each R₃ is independently hydrogen or an optionally substituted alkyl group; and
each R₄ is independently hydrogen, hydrogen, alkyl, alkenyl, alkynyl, aryl, aralkyl, heteroaryl, heteroaralkyl, cycloalkyl, cycloalkenyl, heterocyclyl, or heterocyclylalky, and is optionally substituted with alkyl, alkenyl, alkynyl, alkoxy, alkanoyl, alkylamino, alkylthio, aryl, aryloxy, arylamino, aralkyl, aralkoxy, aralkanoyl, aralkamino, heteroaryl, heteroaryloxy, heteroarylamino, heteroaralkyl, heteroaralkoxy, heteroaralkanoyl, heteroaralkamino, cycloalkyl, cycloalkenyl, cycloalkoxy, cycloalkanoyl, cycloalkamino, heterocyclyl, heterocyclyloxy, heterocyclylamino, heterocyclylalky, heterocyclylalkoxy, heterocyclylalkanoyl, heterocyclylalkamino, hydroxyl, thio, amino, amido, alkanoylamino, aroylamino, aralkanoylamino, alkylcarboxy, alkylcarbonyl, aminocarbonyl, alkylaminocarbonyl, carboxy, carbonate, carbamate, guanidinyl, urea, halo, trihalomethyl, cyano, nitro, phosphoryl, sulfonyl, sulfonamido, or azido.

In some of these embodiments, A is:

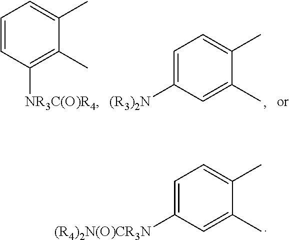

More specifically, A may be:

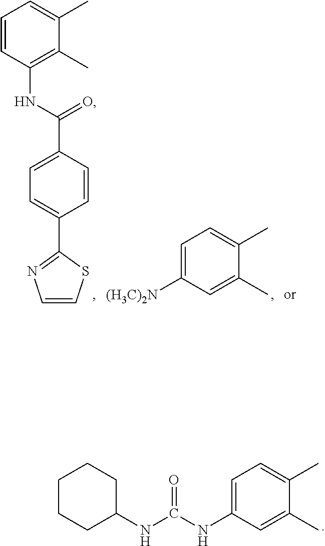

In other more specific embodiments, the compound is represented by structural formula (I); wherein X is O.

In still other more specific embodiments, the compound is represented by structural formula (I); wherein Y is —C(O)NR₁—.

In yet still other more specific embodiments, the compound is represented by structural formula (I); wherein each Z is an optionally substituted C₃ alkylene group.

In some embodiments, the compound is represented by structural formula (I); wherein n is 1.

In more specific embodiments, the compound is represented by structural formula (IA):

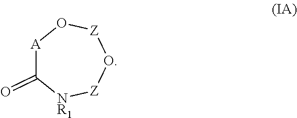

In even more specific embodiments, the compound is represented by structural formula (IA1):

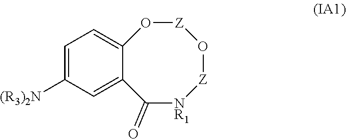

wherein each R₃ is independently hydrogen or an optionally substituted alkyl group.

In some of these embodiments, the compound is represented by structural formula (IA2):

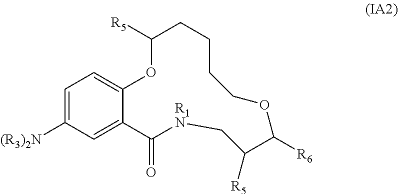

wherein each R₅ is independently an optionally substituted alkyl group; and
R₆ is alkyl, alkenyl, alkynyl, aminoalkyl, aminoalkenyl, aminoalkynyl, aryl, aralkyl, heteroaryl, heteroaralkyl, cycloalkyl, cycloalkenyl, heterocyclyl, or heterocyclylalky, and is optionally substituted with alkyl, alkenyl, alkynyl, alkoxy, alkanoyl, alkylamino, alkylthio, aryl, aryloxy, arylamino, aralkyl, aralkoxy, aralkanoyl, aralkamino, heteroaryl, heteroaryloxy, heteroarylamino, heteroaralkyl, heteroaralkoxy, heteroaralkanoyl, heteroaralkamino, cycloalkyl, cycloalkenyl, cycloalkoxy, cycloalkanoyl, cycloalkamino, heterocyclyl, heterocyclyloxy, heterocyclylamino, heterocyclylalky, heterocyclylalkoxy, heterocyclylalkanoyl, heterocyclylalkamino, hydroxyl, thio, amino, amido, alkanoylamino, aroylamino, aralkanoylamino, alkylcarboxy, alkylcarbonyl, aminocarbonyl, alkylaminocarbonyl, carboxy, carbonate, carbamate, guanidinyl, urea, halo, trihalomethyl, cyano, nitro, phosphoryl, sulfonyl, sulfonamido, or azido.

More specifically, in the compounds of structural formula (IA2),
R₆ is an aminoalkyl group substituted with an arylalkyl group that is optionally substituted with an aryl-substituted amido group.

In other embodiments, the compound is represented by structural formula (I); wherein n is 0. Specifically, in some of these structures, the compound is represented by structural formula (IB):

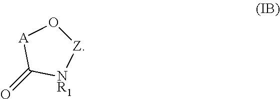

More specifically, the compound is represented by structural formula (IB1):

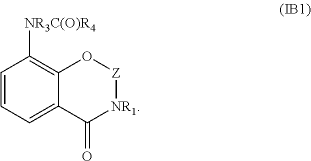

Even more specifically, the compound is represented by structural formula (IB2):

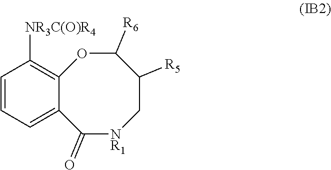

wherein R₅ is an optionally substituted alkyl group; and
R₆ is alkyl, alkenyl, alkynyl, aminoalkyl, aminoalkenyl, aminoalkynyl, aryl, aralkyl, heteroaryl, heteroaralkyl, cycloalkyl, cycloalkenyl, heterocyclyl, or heterocyclylalky, and is optionally substituted with alkyl, alkenyl, alkynyl, alkoxy, alkanoyl, alkylamino, alkylthio, aryl, aryloxy, arylamino, aralkyl, aralkoxy, aralkanoyl, aralkamino, heteroaryl, heteroaryloxy, heteroarylamino, heteroaralkyl, heteroaralkoxy, heteroaralkanoyl, heteroaralkamino, cycloalkyl, cycloalkenyl, cycloalkoxy, cycloalkanoyl, cycloalkamino, heterocyclyl, heterocyclyloxy, heterocyclylamino, heterocyclylalky, heterocyclylalkoxy, heterocyclylalkanoyl, heterocyclylalkamino, hydroxyl, thio, amino, amido, alkanoylamino, aroylamino, aralkanoylamino, alkylcarboxy, alkylcarbonyl, aminocarbonyl, alkylaminocarbonyl, carboxy, carbonate, carbamate, guanidinyl, urea, halo, trihalomethyl, cyano, nitro, phosphoryl, sulfonyl, sulfonamido, or azido.

In some of the compounds of structural formula (IB2), R₆ is an aminoalkyl group substituted with an arylalkyl group that is optionally substituted with an aryl-substituted amido group.

In some embodiments, the compound is represented by structural formula (IC1):

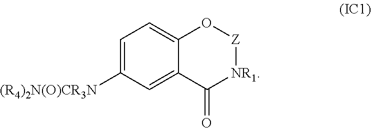

More specifically, the compound is represented by structural formula (IC2):

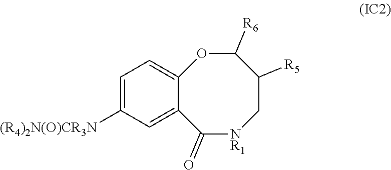

wherein R₅ is an optionally substituted alkyl group; and
R₆ is alkyl, alkenyl, alkynyl, aminoalkyl, aminoalkenyl, aminoalkynyl, aryl, aralkyl, heteroaryl, heteroaralkyl, cycloalkyl, cycloalkenyl, heterocyclyl, or heterocyclylalky, and is optionally substituted with alkyl, alkenyl, alkynyl, alkoxy, alkanoyl, alkylamino, alkylthio, aryl, aryloxy, arylamino, aralkyl, aralkoxy, aralkanoyl, aralkamino, heteroaryl, heteroaryloxy, heteroarylamino, heteroaralkyl, heteroaralkoxy, heteroaralkanoyl, heteroaralkamino, cycloalkyl, cycloalkenyl, cycloalkoxy, cycloalkanoyl, cycloalkamino, heterocyclyl, heterocyclyloxy, heterocyclylamino, heterocyclylalky, heterocyclylalkoxy, heterocyclylalkanoyl, heterocyclylalkamino, hydroxyl, thio, amino, amido, alkanoylamino, aroylamino, aralkanoylamino, alkylcarboxy, alkylcarbonyl, aminocarbonyl, alkylaminocarbonyl, carboxy, carbonate, carbamate, guanidinyl, urea, halo, trihalomethyl, cyano, nitro, phosphoryl, sulfonyl, sulfonamido, or azido.

Even more specifically, in the compound of structural formula (IC2), R₆ is an alkyl group substituted with a sulfonamide group that is optionally substituted with a heteroaryl group.

In other embodiments, the compound of the instant methods of treatment is represented by structural formula (II):

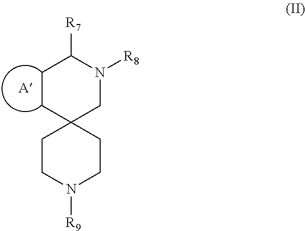

or a stereoisomer, tautomer, or pharmaceutically acceptable salt thereof;
wherein A′ is an optionally substituted aryl or heteroaryl ring system; and
R₇, R₈, and R₉ are each independently, alkyl, alkenyl, alkynyl, acyl, aryl, aralkyl, heteroaryl, heteroaralkyl, cycloalkyl, cycloalkenyl, heterocyclyl, or heterocyclylalky, and is optionally substituted with alkyl, alkenyl, alkynyl, alkoxy, alkanoyl, alkylamino, alkylthio, aryl, aryloxy, arylamino, aralkyl, aralkoxy, aralkanoyl, aralkamino, heteroaryl, heteroaryloxy, heteroarylamino, heteroaralkyl, heteroaralkoxy, heteroaralkanoyl, heteroaralkamino, cycloalkyl, cycloalkenyl, cycloalkoxy, cycloalkanoyl, cycloalkamino, heterocyclyl, heterocyclyloxy, heterocyclylamino, heterocyclylalky, heterocyclylalkoxy, heterocyclylalkanoyl, heterocyclylalkamino, hydroxyl, thio, amino, amido, alkanoylamino, aroylamino, aralkanoylamino, alkylcarboxy, alkylcarbonyl, aminocarbonyl, alkylaminocarbonyl, carboxy, carbonate, carbamate, guanidinyl, urea, halo, trihalomethyl, cyano, nitro, phosphoryl, sulfonyl, sulfonamido, or azido.

In more specific embodiments, the compound is represented by structural formula (II); wherein A′ is an optionally substituted bicyclic aryl or heteroaryl ring system.

In other more specific embodiments, the compound is represented by structural formula (II); wherein R₇ is an alkyl group optionally substituted with hydroxyl, thio, amino, carboxy, carbonate, carbamate, guanidinyl, urea, halo, trihalomethyl, cyano, nitro, phosphoryl, sulfonyl, sulfonamido, or azido.

In still other more specific embodiments, the compound is represented by structural formula (II); wherein R₈ is an aryl, aralkyl, heteroaryl, heteroaralkyl group optionally substituted with hydroxyl, thio, amino, carboxy, carbonate, carbamate, guanidinyl, urea, halo, trihalomethyl, cyano, nitro, phosphoryl, sulfonyl, sulfonamido, or azido.

In even other more specific embodiments, the compound is represented by structural formula (II); wherein R₉ is an acyl group optionally substituted with hydroxyl, thio, amino, carboxy, carbonate, carbamate, guanidinyl, urea, halo, trihalomethyl, cyano, nitro, phosphoryl, sulfonyl, sulfonamido, or azido.

In some embodiments, the compound is represented by structural formula (IIA):

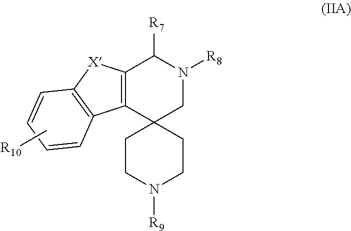

wherein X′ is N—R₁₁, O, or S;
R₁₀ is an alkoxy, alkanoyl, alkylamino, or alkylthio group; and
R₁₁ is hydrogen or alkyl.

More specifically, the compound is represented by structural formula (IIA);
wherein R₇ is an alkyl group optionally substituted with hydroxyl, thio, amino, carboxy, carbonate, carbamate, guanidinyl, urea, halo, trihalomethyl, cyano, nitro, phosphoryl, sulfonyl, sulfonamido, or azido;
R₈ is an aryl, aralkyl, heteroaryl, heteroaralkyl group optionally substituted with hydroxyl, thio, amino, carboxy, carbonate, carbamate, guanidinyl, urea, halo, trihalomethyl, cyano, nitro, phosphoryl, sulfonyl, sulfonamido, or azido; and
R₉ is an acyl group optionally substituted with hydroxyl, thio, amino, carboxy, carbonate, carbamate, guanidinyl, urea, halo, trihalomethyl, cyano, nitro, phosphoryl, sulfonyl, sulfonamido, or azido.

In specific embodiments, the compound used in the instant methods is one of the following compounds:

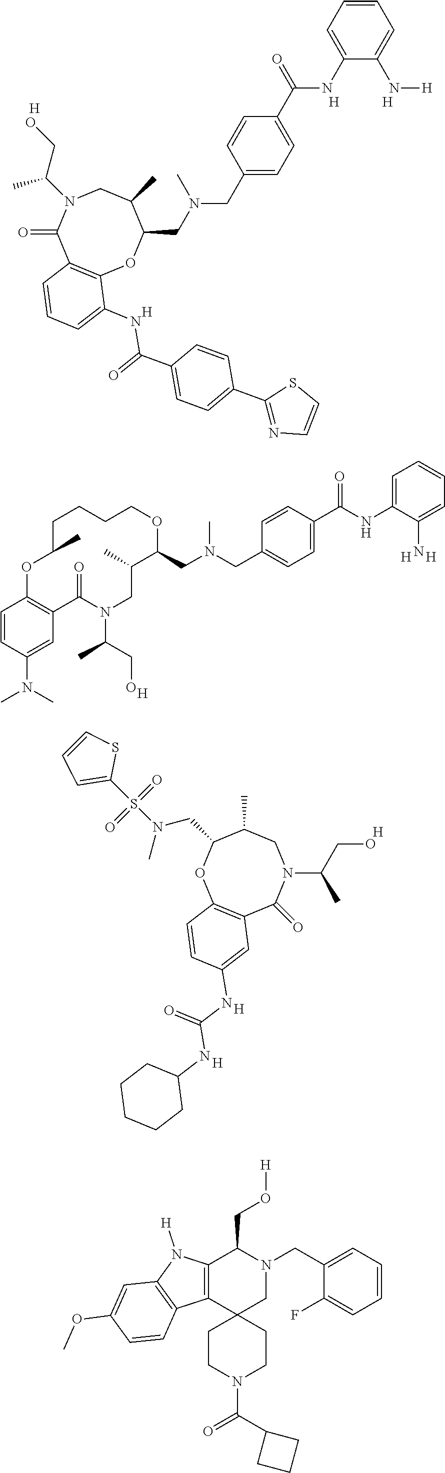

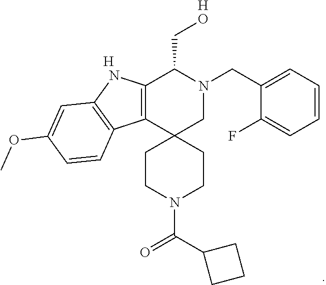

In the instant methods of treatment, the therapeutic compound is administered at a dose sufficient to achieve a desired endpoint, for example the remission of synovial sarcoma tumors in the subject. Administered dosages for the therapeutic compound are in accordance with dosages and scheduling regimens practiced by those of skill in the art. General guidance for appropriate dosages of pharmacological agents used in the present methods is provided in Goodman and Gilman's The Pharmacological Basis of Therapeutics, 12th Edition (2010), and in Physicians' Desk Reference, 69^{th} Edition (2015), each of which is hereby incorporated herein by reference.

The appropriate dosage of a particular therapeutic compound will vary according to several factors, including the chosen route of administration, the formulation of the composition, patient response, the severity of the condition, the subject's weight, the susceptibility of the subject to side effects, and the judgment of the prescribing physician. The dosage may be increased or decreased over time, as required by an individual subject. Preferred dosages for a given compound are readily determinable by those of ordinary skill in the art by a variety of means. Dosage amount and interval may be adjusted individually to provide plasma levels of the active compounds which are sufficient to maintain a desired therapeutic effect.

In one embodiment, the therapeutic compound is administered in an amount of about 1 μg to 1000 mg per dose, e.g., about 1 μg to 5 μg, about 5 μg to 10 μg, about 10 μg to 50 μg, about 50 μg to 100 μg, about 100 μg to 200 μg, about 200 μg to 400 μg, about 400 μg to 800 μg, about 800 μg to 1 mg, about 1 mg to 2 mg, about 2 mg to 4 mg, about 4 mg to 8 mg, about 8 mg to 10 mg, about 10 mg to 20 mg, about 20 mg to 40 mg, about 40 mg to 80 mg, about 80 mg to 100 mg, about 100 mg to 2000 mg, about 200 mg to 400 mg, about 400 mg to 1000 mg per dose, or even higher.

In another embodiment, the amount of the therapeutic compound administered per dose is determined on a per body weight basis. For example, the amount of the compound or composition per dose, as determined on a per body weight basis, may be, for example, about 10 ng/kg, about 15 ng/kg, about 20 ng/kg, about 50 ng/kg, about 100 ng/kg, about 200 ng/kg, about 500 ng/kg, about 1 mg/kg, about 2 mg/kg, about 5 mg/kg, about 10 μg/kg, about 20 mg/kg, about 50 mg/kg, about 100 mg/kg, about 200 mg/kg, about 500 mg/kg, about 1 mg/kg, about 2 mg/kg, about 5 mg/kg, about 10 mg/kg, or even higher.

In an embodiment, multiple doses of the therapeutic compound are administered. The frequency of administration of the compound may vary depending on any of a variety of factors, e.g., severity of the symptoms, and the like. For example, in an embodiment, the compound is administered once per month, twice per month, three times per month, every other week (qow), once per week (qw), twice per week (biw), three times per week (tiw), four times per week, five times per week, six times per week, every other day (qod), daily (qd), twice a day (bid), or three times a day (tid). In some embodiments, for example during a surgical procedure, the compound may be administered even more frequently. For example, the compound may be administered at least once per four hours, at least once per two hours, at least once per hour, at least twice per hour, at least four times per hour, at least 10 times per hour, or even more frequently.

In some embodiments, the compound is administered continuously. The duration of administration of the therapeutic compound, e.g., the period of time over which the compound is administered, may vary, depending on any of a variety of factors, e.g., the chosen route of administration, the formulation of the composition, patient response, and so forth. For example, the compound may be administered over a period of time of at least 5 minutes, at least 30 minutes, at least one hour, at least 2 hours, at least 4 hours, at least 8 hours, at least one day, at least one week, or even longer. In other embodiments, the compound may be administered over a period of time of no more than one week, no more than one day, no more than 8 hours, no more than 4 hours, no more than 2 hours, no more than one hour, no more than 30 minutes, no more than 5 minutes, or even shorter. In some embodiments, the compound may be administered for a time period of about 5 minutes to 30 minutes, of about 30 minutes to one hour, of about one hour to 2 hours, of about 2 hours to 4 hours, of about 4 hours to 8 hours, of about 8 hours to one day, or of about one day to one week.

In various embodiments, the therapeutic compounds of the instant disclosure are delivered to the subject via injection. The compounds are preferably formulated, as described below, in compositions that facilitate the effective delivery of the injected compounds to the target tissue. In particular, and as described below, the compositions are preferably delivered in the instant methods of treatment by parenteral administration, as is well understood in the art.

### Compounds

According to another aspect of the invention, novel compounds are provided that are effective in the treatment of synovial sarcoma in animal subjects. A first category of compounds is represented by structural formula (I):

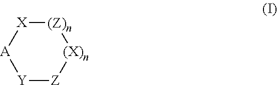

or a stereoisomer, tautomer, or pharmaceutically acceptable salt thereof;
wherein A is an optionally substituted bivalent aryl or heteroaryl group;
each X is independently O or S;
Y is —C(O)NR₁— or —C(S)NR₁—, wherein R₁ is hydrogen, alkyl, alkenyl, alkynyl, aryl, aralkyl, heteroaryl, heteroaralkyl, cycloalkyl, cycloalkenyl, heterocyclyl, or heterocyclylalky, and is optionally substituted with alkyl, alkenyl, alkynyl, alkoxy, alkanoyl, alkylamino, alkylthio, aryl, aryloxy, arylamino, aralkyl, aralkoxy, aralkanoyl, aralkamino, heteroaryl, heteroaryloxy, heteroarylamino, heteroaralkyl, heteroaralkoxy, heteroaralkanoyl, heteroaralkamino, cycloalkyl, cycloalkenyl, cycloalkoxy, cycloalkanoyl, cycloalkamino, heterocyclyl, heterocyclyloxy, heterocyclylamino, heterocyclylalky, heterocyclylalkoxy, heterocyclylalkanoyl, heterocyclylalkamino, hydroxyl, thio, amino, amido, alkanoylamino, aroylamino, aralkanoylamino, alkylcarboxy, alkylcarbonyl, aminocarbonyl, alkylaminocarbonyl, carboxy, carbonate, carbamate, guanidinyl, urea, halo, trihalomethyl, cyano, nitro, phosphoryl, sulfonyl, sulfonamido, or azido;
each Z is independently an optionally substituted C₂₋₆ alkylene group; and
each n is independently either 0 or 1;
provided that the compound is not one of the following compounds:

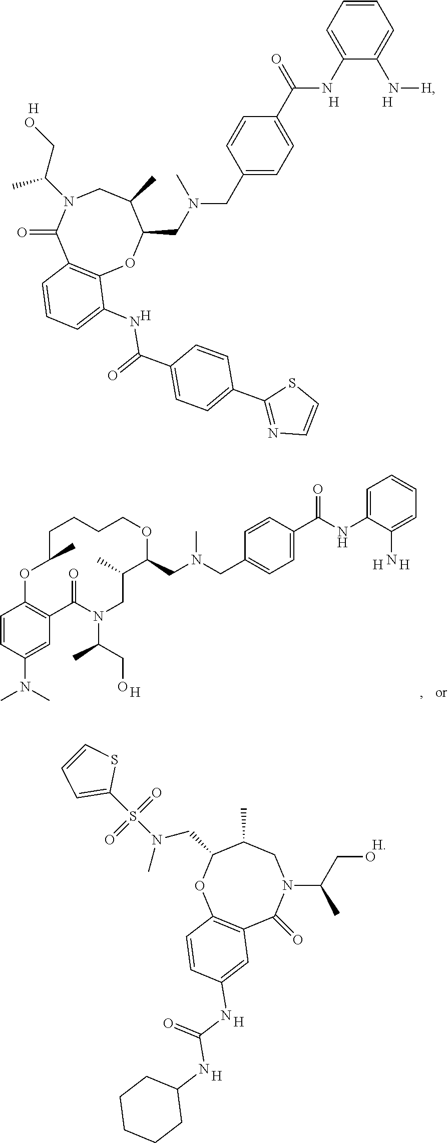

In more specific embodiments of the compound,
A is

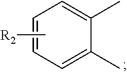

R₂ is hydrogen, —N(R₃)₂, —NR₃C(O)R₄, or —NR₃C(O)N(R₄)₂;
each R₃ is independently hydrogen or an optionally substituted alkyl group; and
each R₄ is independently hydrogen, hydrogen, alkyl, alkenyl, alkynyl, aryl, aralkyl, heteroaryl, heteroaralkyl, cycloalkyl, cycloalkenyl, heterocyclyl, or heterocyclylalky, and is optionally substituted with alkyl, alkenyl, alkynyl, alkoxy, alkanoyl, alkylamino, alkylthio, aryl, aryloxy, arylamino, aralkyl, aralkoxy, aralkanoyl, aralkamino, heteroaryl, heteroaryloxy, heteroarylamino, heteroaralkyl, heteroaralkoxy, heteroaralkanoyl, heteroaralkamino, cycloalkyl, cycloalkenyl, cycloalkoxy, cycloalkanoyl, cycloalkamino, heterocyclyl, heterocyclyloxy, heterocyclylamino, heterocyclylalky, heterocyclylalkoxy, heterocyclylalkanoyl, heterocyclylalkamino, hydroxyl, thio, amino, amido, alkanoylamino, aroylamino, aralkanoylamino, alkylcarboxy, alkylcarbonyl, aminocarbonyl, alkylaminocarbonyl, carboxy, carbonate, carbamate, guanidinyl, urea, halo, trihalomethyl, cyano, nitro, phosphoryl, sulfonyl, sulfonamido, or azido.

In some of these embodiments, A is:

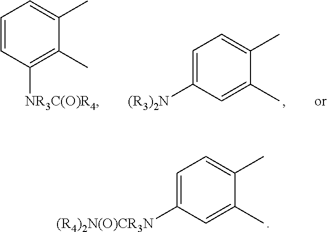

More specifically, A may be:

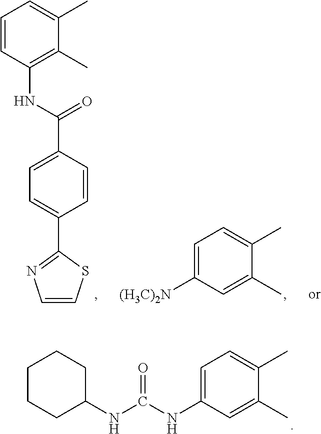

In other more specific compounds of structural formula (I), X is O.

In still other more specific compounds of structural formula (I), Y is —C(O)NR₁—.

In yet still other more specific compounds of structural formula (I), each Z is an optionally substituted C₃ alkylene group.

In some compounds of structural formula (I), n is 1, and in more specific embodiments, the compound is represented by structural formula (IA):

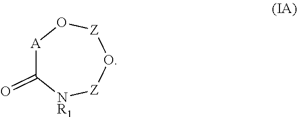

In even more specific embodiments, the compound is represented by structural formula (IA1):

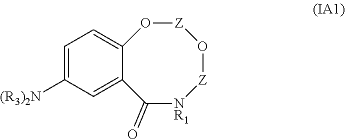

wherein each R₃ is independently hydrogen or an optionally substituted alkyl group.

In some of these embodiments, the compound is represented by structural formula (IA2):

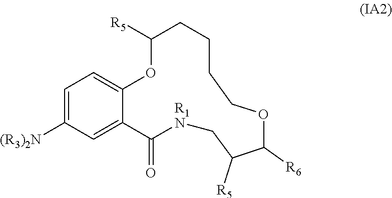

wherein each R₅ is independently an optionally substituted alkyl group; and
R₆ is alkyl, alkenyl, alkynyl, aminoalkyl, aminoalkenyl, aminoalkynyl, aryl, aralkyl, heteroaryl, heteroaralkyl, cycloalkyl, cycloalkenyl, heterocyclyl, or heterocyclylalky, and is optionally substituted with alkyl, alkenyl, alkynyl, alkoxy, alkanoyl, alkylamino, alkylthio, aryl, aryloxy, arylamino, aralkyl, aralkoxy, aralkanoyl, aralkamino, heteroaryl, heteroaryloxy, heteroarylamino, heteroaralkyl, heteroaralkoxy, heteroaralkanoyl, heteroaralkamino, cycloalkyl, cycloalkenyl, cycloalkoxy, cycloalkanoyl, cycloalkamino, heterocyclyl, heterocyclyloxy, heterocyclylamino, heterocyclylalky, heterocyclylalkoxy, heterocyclylalkanoyl, heterocyclylalkamino, hydroxyl, thio, amino, amido, alkanoylamino, aroylamino, aralkanoylamino, alkylcarboxy, alkylcarbonyl, aminocarbonyl, alkylaminocarbonyl, carboxy, carbonate, carbamate, guanidinyl, urea, halo, trihalomethyl, cyano, nitro, phosphoryl, sulfonyl, sulfonamido, or azido.

More specifically, in the compounds of structural formula (IA2),
R₆ is an aminoalkyl group substituted with an arylalkyl group that is optionally substituted with an aryl-substituted amido group.

In other embodiments of the compound of structural formula (I), n is 0. Specifically, in some of these structures, the compound is represented by structural formula (IB):

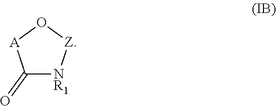

More specifically, the compound is represented by structural formula (IB1):

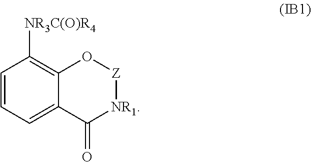

Even more specifically, the compound is represented by structural formula (IB2):

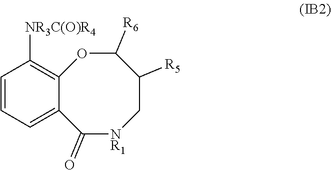

wherein R₅ is an optionally substituted alkyl group; and
R₆ is alkyl, alkenyl, alkynyl, aminoalkyl, aminoalkenyl, aminoalkynyl, aryl, aralkyl, heteroaryl, heteroaralkyl, cycloalkyl, cycloalkenyl, heterocyclyl, or heterocyclylalky, and is optionally substituted with alkyl, alkenyl, alkynyl, alkoxy, alkanoyl, alkylamino, alkylthio, aryl, aryloxy, arylamino, aralkyl, aralkoxy, aralkanoyl, aralkamino, heteroaryl, heteroaryloxy, heteroarylamino, heteroaralkyl, heteroaralkoxy, heteroaralkanoyl, heteroaralkamino, cycloalkyl, cycloalkenyl, cycloalkoxy, cycloalkanoyl, cycloalkamino, heterocyclyl, heterocyclyloxy, heterocyclylamino, heterocyclylalky, heterocyclylalkoxy, heterocyclylalkanoyl, heterocyclylalkamino, hydroxyl, thio, amino, amido, alkanoylamino, aroylamino, aralkanoylamino, alkylcarboxy, alkylcarbonyl, aminocarbonyl, alkylaminocarbonyl, carboxy, carbonate, carbamate, guanidinyl, urea, halo, trihalomethyl, cyano, nitro, phosphoryl, sulfonyl, sulfonamido, or azido.

In some of the compounds of structural formula (IB2), R₆ is an aminoalkyl group substituted with an arylalkyl group that is optionally substituted with an aryl-substituted amido group.

In some of the structures of structural formula (IB), the compound is represented by structural formula (IC1):

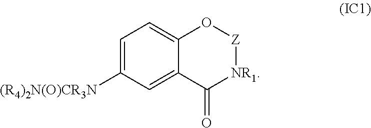

More specifically, the compound is represented by structural formula (IC2):

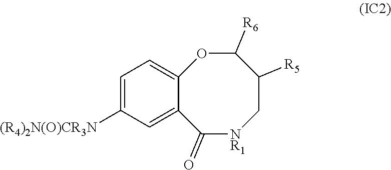

wherein R₅ is an optionally substituted alkyl group; and
R₆ is alkyl, alkenyl, alkynyl, aminoalkyl, aminoalkenyl, aminoalkynyl, aryl, aralkyl, heteroaryl, heteroaralkyl, cycloalkyl, cycloalkenyl, heterocyclyl, or heterocyclylalky, and is optionally substituted with alkyl, alkenyl, alkynyl, alkoxy, alkanoyl, alkylamino, alkylthio, aryl, aryloxy, arylamino, aralkyl, aralkoxy, aralkanoyl, aralkamino, heteroaryl, heteroaryloxy, heteroarylamino, heteroaralkyl, heteroaralkoxy, heteroaralkanoyl, heteroaralkamino, cycloalkyl, cycloalkenyl, cycloalkoxy, cycloalkanoyl, cycloalkamino, heterocyclyl, heterocyclyloxy, heterocyclylamino, heterocyclylalky, heterocyclylalkoxy, heterocyclylalkanoyl, heterocyclylalkamino, hydroxyl, thio, amino, amido, alkanoylamino, aroylamino, aralkanoylamino, alkylcarboxy, alkylcarbonyl, aminocarbonyl, alkylaminocarbonyl, carboxy, carbonate, carbamate, guanidinyl, urea, halo, trihalomethyl, cyano, nitro, phosphoryl, sulfonyl, sulfonamido, or azido.

Even more specifically, in the compound of structural formula (IC2), R₆ is an alkyl group substituted with a sulfonamide group that is optionally substituted with a heteroaryl group.

A second category of compounds is represented by structural formula (II):

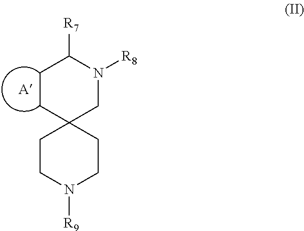

or a stereoisomer, tautomer, or pharmaceutically acceptable salt thereof;
wherein A′ is an optionally substituted aryl or heteroaryl ring system; and
R₇, R₈, and R₉ are each independently, alkyl, alkenyl, alkynyl, acyl, aryl, aralkyl, heteroaryl, heteroaralkyl, cycloalkyl, cycloalkenyl, heterocyclyl, or heterocyclylalky, and is optionally substituted with alkyl, alkenyl, alkynyl, alkoxy, alkanoyl, alkylamino, alkylthio, aryl, aryloxy, arylamino, aralkyl, aralkoxy, aralkanoyl, aralkamino, heteroaryl, heteroaryloxy, heteroarylamino, heteroaralkyl, heteroaralkoxy, heteroaralkanoyl, heteroaralkamino, cycloalkyl, cycloalkenyl, cycloalkoxy, cycloalkanoyl, cycloalkamino, heterocyclyl, heterocyclyloxy, heterocyclylamino, heterocyclylalky, heterocyclylalkoxy, heterocyclylalkanoyl, heterocyclylalkamino, hydroxyl, thio, amino, amido, alkanoylamino, aroylamino, aralkanoylamino, alkylcarboxy, alkylcarbonyl, aminocarbonyl, alkylaminocarbonyl, carboxy, carbonate, carbamate, guanidinyl, urea, halo, trihalomethyl, cyano, nitro, phosphoryl, sulfonyl, sulfonamido, or azido;
provided that the compound is not one of the following compounds:

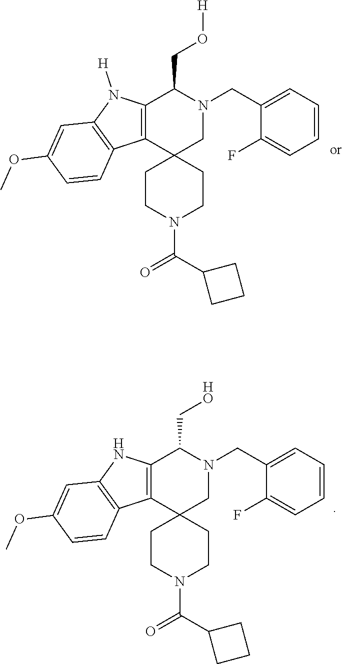 [SMILES: COc1ccc2c3c([nH]c2c1)[C@@H](CO)N(Cc1ccccc1F)CC31CCN(C(=O)C2CCC2)CC1 ; COc1ccc2c3c([nH]c2c1)[C@H](CO)N(Cc1ccccc1F)CC31CCN(C(=O)C2CCC2)CC1]

In more specific embodiments of the compound of structural formula (II), A′ is an optionally substituted bicyclic aryl or heteroaryl ring system.

In other more specific embodiments of the compound of structural formula (II), R₇ is an alkyl group optionally substituted with hydroxyl, thio, amino, carboxy, carbonate, carbamate, guanidinyl, urea, halo, trihalomethyl, cyano, nitro, phosphoryl, sulfonyl, sulfonamido, or azido.

In still other more specific embodiments of the compound of structural formula (II), R₈ is an aryl, aralkyl, heteroaryl, heteroaralkyl group optionally substituted with hydroxyl, thio, amino, carboxy, carbonate, carbamate, guanidinyl, urea, halo, trihalomethyl, cyano, nitro, phosphoryl, sulfonyl, sulfonamido, or azido.

In even other more specific embodiments of the compound of structural formula (II), R₉ is an acyl group optionally substituted with hydroxyl, thio, amino, carboxy, carbonate, carbamate, guanidinyl, urea, halo, trihalomethyl, cyano, nitro, phosphoryl, sulfonyl, sulfonamido, or azido.

In some embodiments, the compound is represented by structural formula (IIA):

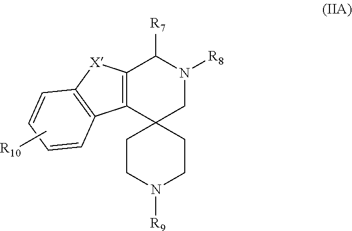

wherein X′ is N—R₁₁, O, or S;
R₁₀ is an alkoxy, alkanoyl, alkylamino, or alkylthio group; and
R₁₁ is hydrogen or alkyl.

More specifically, in the compound of structural formula (IIA),
R₇ is an alkyl group optionally substituted with hydroxyl, thio, amino, carboxy, carbonate, carbamate, guanidinyl, urea, halo, trihalomethyl, cyano, nitro, phosphoryl, sulfonyl, sulfonamido, or azido;
R₈ is an aryl, aralkyl, heteroaryl, heteroaralkyl group optionally substituted with hydroxyl, thio, amino, carboxy, carbonate, carbamate, guanidinyl, urea, halo, trihalomethyl, cyano, nitro, phosphoryl, sulfonyl, sulfonamido, or azido; and
R₉ is an acyl group optionally substituted with hydroxyl, thio, amino, carboxy, carbonate, carbamate, guanidinyl, urea, halo, trihalomethyl, cyano, nitro, phosphoryl, sulfonyl, sulfonamido, or azido.

As used herein, the term “alkyl” refers to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In some embodiments, a straight chain or branched chain alkyl has 30 or fewer carbon atoms in its backbone (e.g., C₁-C₃₀ for straight chains, C₃-C₃₀ for branched or cyclic chains), more specifically 20 or fewer carbon atoms in its backbone (e.g., C₁-C₂₀ for straight chains, C₃-C₂₀ for branched or cyclic chains), and even more specifically 10 or fewer carbon atoms in its backbone (e.g., C₁-C₁₀ for straight chains, C₃-C₁₀ for branched or cyclic chains). Likewise, some cycloalkyls have from 3-10 carbon atoms in their ring structure, and more specifically have 5, 6 or 7 carbons in the ring structure.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents may include, for example, a halo, a hydroxyl, a carbonyl (such as a keto, a carboxy, an alkoxycarbonyl, a formyl, an acyl, a carbonate, a carbamate, an ester, or a urea), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a thio, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN, halo, and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, halo, and the like.

The term “alkylene”, as used herein, refers to the bivalent radical of saturated aliphatic groups, including bivalent radicals of all of the above alkyl groups.

As used herein, the term “alkoxy” refers to an oxy group substituted with an alkyl group, in certain specific embodiments, a lower alkyl group. Representative alkoxy groups include methoxy, ethoxy, propoxy, t-butoxy, and the like.

The term “alkenyl”, as used herein, refers to an aliphatic group containing at least one double bond and is intended to include both “unsubstituted alkenyls” and “substituted alkenyls”, the latter of which refers to alkenyl moieties having substituents replacing a hydrogen on one or more carbons of the alkenyl group.

Such substituents may occur on one or more carbons that are included or not included in one or more double bonds. Moreover, such substituents include all those contemplated for alkyl groups, as discussed above, except where stability is prohibitive. For example, substitution of alkenyl groups by one or more alkyl, cycloalkyl, heterocyclyl, aryl, or heteroaryl groups is contemplated.

As used herein, the term “acyl” refers to the group

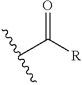

wherein R represents a hydrogen or hydrocarbyl group.

The term “C_{x-y}” when used in conjunction with a chemical moiety, such as acyl, acyloxy, alkyl, alkenyl, alkynyl, or alkoxy, is meant to include groups that contain from x to y carbons in the chain. For example, the term “C_{x-y}-alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-trifluoroethyl, etc. “C₀-alkyl” indicates a hydrogen where the group is in a terminal position, or is a bond if internal. The terms “C_{2-y}-alkenyl” and “C_{2-y}-alkynyl” refer to substituted or unsubstituted unsaturated aliphatic groups analogous in length and possible substitution to the alkyls described above, but that contain at least one double or triple bond, respectively.

The term “alkylamino”, as used herein, refers to an amino group substituted with at least one alkyl group.

The term “alkylthio”, as used herein, refers to a thiol group substituted with an alkyl group and may be represented by the general formula alkyl-S—.

The term “alkynyl”, as used herein, refers to an aliphatic group containing at least one triple bond and is intended to include both “unsubstituted alkynyls” and “substituted alkynyls”, the latter of which refers to alkynyl moieties having substituents replacing a hydrogen on one or more carbons of the alkynyl group. Such substituents may occur on one or more carbons that are included or not included in one or more triple bonds. Moreover, such substituents include all those contemplated for alkyl groups, as discussed above, except where stability is prohibitive. For example, substitution of alkynyl groups by one or more alkyl, cycloalkyl, heterocyclyl, aryl, or heteroaryl groups is contemplated.

The term “amide” or “amido”, as used herein, refers to a group

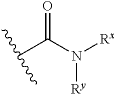

wherein R^{x} and R^{y} each independently represent a hydrogen or hydrocarbyl group, or R^{x} and R^{y} taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

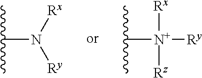

wherein R^{x}, R^{y}, and R^{z} each independently represent a hydrogen or a hydrocarbyl group, or R^{x} and R^{y} taken together with the N atom to which they are attached complete a heterocycle having from 4 to 8 atoms in the ring structure.

The terms “aminoalkyl”, “aminoalkenyl”, and “aminoalkynyl”, as used herein, refer to an alkyl group, an alkenyl group, or an alkynyl group, respectively, substituted with an amino group.

The term “aralkyl”, as used herein, refers to an alkyl group substituted with an aryl group.

The term “aryl” as used herein includes substituted or unsubstituted single-ring aromatic groups in which each atom of the ring is carbon. In certain embodiments, the ring is a 5- to 7-membered ring, and in more specific embodiments is a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

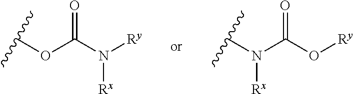

wherein R^{x} and R^{y} independently represent hydrogen or a hydrocarbyl group, or R^{x} and R^{y} taken together with the atoms to which they are attached complete a heterocycle having from 4 to 8 atoms in the ring structure.

The term “cycloalkyl”, as used herein, refers to a non-aromatic saturated or unsaturated ring in which each atom of the ring is carbon. In certain embodiments, a cycloalkyl ring contains from 3 to 10 atoms, and in more specific embodiments from 5 to 7 atoms.

The term “carbonate” is art-recognized and refers to a group —OCO₂—R^{x}, wherein R^{x} represents a hydrocarbyl group.

The term “carboxy”, as used herein, refers to a group represented by the formula —CO₂H.

The term “ester”, as used herein, refers to a group —C(O)OR^{x} wherein R^{x} represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The term “guanidinyl” is art-recognized and may be represented by the general formula

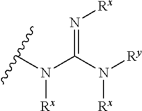

wherein R^{x} and R^{y} independently represent hydrogen or a hydrocarbyl.

The terms “halo” and “halogen” as used herein mean halogen and include chloro, fluoro, bromo, and iodo.

The terms “hetaralkyl” and “heteroaralkyl”, as used herein, refer to an alkyl group substituted with a hetaryl group.

The terms “heteroaryl” and “hetaryl” include substituted or unsubstituted aromatic single ring structures, in certain specific embodiments 5- to 7-membered rings, more specifically 5- to 6-membered rings, whose ring structures include at least one heteroatom, in some embodiments one to four heteroatoms, and in more specific embodiments one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Typical heteroatoms are nitrogen, oxygen, and sulfur.

The terms “heterocyclyl”, “heterocycle”, and “heterocyclic” refer to substituted or unsubstituted non-aromatic ring structures, in certain specific embodiments 3- to 10-membered rings, more specifically 3- to 7-membered rings, whose ring structures include at least one heteroatom, in some embodiments one to four heteroatoms, and in more specific embodiments one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “heterocyclylalkyl”, as used herein, refers to an alkyl group substituted with a heterocycle group.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and trifluoromethyl are considered to be hydrocarbyl for the purposes herein, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer non-hydrogen atoms in the substituent, and in certain embodiments, six or fewer. A “lower alkyl”, for example, refers to an alkyl group that contains ten or fewer carbon atoms, and in specific embodiments six or fewer carbon atoms. In certain embodiments, the acyl, acyloxy, alkyl, alkenyl, alkynyl, and alkoxy substituents defined herein are respectively lower acyl, lower acyloxy, lower alkyl, lower alkenyl, lower alkynyl, and lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, more specifically from 5 to 7.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc., under conditions in which the compound is to be used. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents may include any substituents described herein, for example, a halogen, a hydroxyl, a carbonyl (such as a keto, a carboxy, an alkoxycarbonyl, a formyl, an acyl, a carbonate, a carbamate, an ester, or a urea), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain may themselves be substituted, if appropriate.

Unless specifically described as “unsubstituted”, references to chemical moieties herein are understood to include substituted variants. For example, reference to an “aryl” group or moiety implicitly includes both substituted and unsubstituted variants.

The term “sulfate” is art-recognized and refers to the group —OSO3H, or a pharmaceutically acceptable salt thereof.

The term “sulfonamide” is art-recognized and refers to the group represented by the general formulae

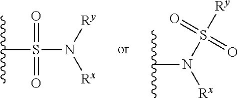

wherein R^{x} and R^{y} independently represent hydrogen or hydrocarbyl.

The term “sulfoxide” is art-recognized and refers to the group —S(O)—R^{x}, wherein R^{x} represents a hydrocarbyl.

The term “sulfonate” is art-recognized and refers to the group —SO₃H, or a pharmaceutically acceptable salt thereof.

The term “sulfone” is art-recognized and refers to the group —S(O)₂—R^{x}, wherein R^{x} represents a hydrocarbyl.

The term “thioalkyl”, as used herein, refers to an alkyl group substituted with a thiol group.

The term “thioester”, as used herein, refers to a group —C(O)SR^{x} or —SC(O)R^{x} wherein R^{x} represents a hydrocarbyl.

The term “thioether”, as used herein, is equivalent to an ether, wherein the oxygen is replaced with a sulfur.

The term “urea” is art-recognized and may be represented by the general formula

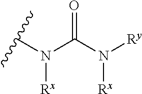

wherein R^{x} and R^{y} independently represent hydrogen or a hydrocarbyl.

It should be understood that some of the compounds of the invention may contain one or more stereocenters, and that, absent an explicit indication otherwise, compounds containing only one or the other stereoisomer at the stereocenter, or a mixture of the stereoisomers, in any combination, are considered within the scope of the invention. For example, the compounds of the invention may be pure enantiomeric or diastereomeric forms of a given molecule or may be mixtures of the enantiomeric or diastereomic forms, at any ratio.

### Pharmaceutical Compositions

In another aspect, the instant invention provides pharmaceutical compositions comprising a compound of the invention and a pharmaceutically acceptable carrier.

Pharmaceutically acceptable carriers are well known in the art and include, for example, aqueous solutions such as water or physiologically buffered saline or other solvents or vehicles such as glycols, glycerol, oils such as olive oil, injectable organic esters, lipid emulsions such as intralipid and the like, and other suitable carriers. In a specific embodiment, when such pharmaceutical compositions are for human administration, the aqueous solution is pyrogen free, or substantially pyrogen free. The excipients may be chosen, for example, to effect delayed release of an agent or to selectively target one or more cells, tissues or organs. The pharmaceutical composition may be in dosage unit form such as tablet, capsule, sprinkle capsule, granule, powder, syrup, suppository, injection or the like. The composition may also be present in a transdermal delivery system, e.g., a skin patch.

A pharmaceutically acceptable carrier may contain physiologically acceptable agents that act, for example, to stabilize or to increase the absorption of a compound of the instant invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The pharmaceutical composition also may comprise a liposome or other polymer matrix, which may have incorporated therein, for example, a compound of the invention. Liposomes, for example, which consist of phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms that are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition, or vehicle, such as a liquid or solid filler, diluent, excipient, solvent, or encapsulating material, involved in carrying or transporting the subject compounds from one organ, or portion of the body, to another organ, or portion of the body. Each carrier must be “acceptable” in the sense of being compatible with the other ingredients of the formulation and not injurious to the patient. Some examples of materials that can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. See Remington: The Science and Practice of Pharmacy, 22nd ed. (Allen et al., eds.), 2012.

A pharmaceutical composition containing a compound of the instant invention may be administered to a subject by any of a number of routes of administration including, for example, orally (for example, drenches as in aqueous or non-aqueous solutions or suspensions, tablets, boluses, powders, granules, pastes for application to the tongue); sublingually; anally; rectally; or vaginally (for example, as a pessary, cream, or foam); parenterally (including intramuscularly, intravenously, subcutaneously, or intrathecally as, for example, a sterile solution or suspension); nasally; intraperitoneally; subcutaneously; transdermally (for example as a patch applied to the skin); or topically (for example, as a cream, ointment or spray applied to the skin). In certain embodiments, a compound of the instant invention may be simply dissolved or suspended in sterile water. Details of appropriate routes of administration and compositions suitable for same can be found in, for example, U.S. Pat. Nos. 6,110,973; 5,763,493; 5,731,000; 5,541,231; 5,427,798; 5,358,970; and 4,172,896, as well as in patents cited therein.

The formulations of the present invention may conveniently be presented in unit dosage form and may be prepared by any methods well known in the art of pharmacy. The amount of active ingredient that may be combined with a carrier material to produce a single dosage form will vary depending upon the subject being treated and the particular mode of administration. The amount of active ingredient that may be combined with a carrier material to produce a single dosage form will generally be that amount of the compound that produces a therapeutic effect. Generally, out of one hundred percent, this amount will range from about 0.1 percent to about 50 percent of active ingredient, in some embodiments from about 0.2 percent to about 10 percent, and in more specific embodiments from about 0.5 percent to about 2 percent. For example, compounds of the present disclosure may be formulated in a unit dose form between about 1 μg and 1000 mg. In some embodiments, compounds or compositions of the present disclosure may be formulated in a unit dose of about 1 μg to 20 μg, of about 20 μg to 1 mg, of about 1 mg to 10 mg, of about 10 mg to 100 mg, and of about 50 mg to 500 mg. In particular, an embodiment including a compound may be formulated in 0.1 μg, 0.2 μg, 0.5 μg, 1 μg, 20 μg, 50 μg, 100 μg, 200 μg, 500 μg, 1 mg, 2 mg, 5 mg, 10 mg, 20 mg, 50 mg, 100 mg, 200 mg, and 500 mg unit dose form.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary, or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that may be used include polymeric substances and waxes. The active ingredient may also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions may also include adjuvants such as wetting agents, emulsifying and suspending agents, sweetening, flavoring, coloring, perfuming, and preservative agents.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, and intrasternal injection and infusion.

Pharmaceutical compositions of this invention suitable for parenteral administration comprise one or more compounds of the invention in combination with one or more pharmaceutically acceptable sterile isotonic aqueous or nonaqueous solutions, dispersions, suspensions, or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, injectable organic esters, such as ethyl oleate, and lipid emulsions, such as Intralipid and the like. Proper fluidity may be maintained, for example, by the use of coating materials, such as lecithin, by the maintenance of the required particle size in the case of dispersions, and by the use of surfactants.

These compositions may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid, chelators and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsuled matrices of the subject compounds in biodegradable polymers such as polylactide-polyglycolide. Depending on the ratio of drug to polymer, and the nature of the particular polymer employed, the rate of drug release may be controlled. Examples of other biodegradable polymers include poly(orthoesters) and poly(anhydrides). Depot injectable formulations are also prepared by entrapping the drug in liposomes or microemulsions that are compatible with body tissue.

Methods of introduction may also be provided by rechargeable or biodegradable devices. Various slow release polymeric devices have been developed and tested in vivo in recent years for the controlled delivery of drugs. A variety of biocompatible polymers (including hydrogels), including both biodegradable and non-degradable polymers, may be used to form an implant for the sustained release of a compound at a particular target site.

In another aspect, the invention is provided substantially as described in any part of the instant disclosure, including the examples, in any combination, and as shown in the accompanying drawings.

It will be readily apparent to one of ordinary skill in the relevant arts that other suitable modifications and adaptations to the methods, compounds, and compositions described herein may be made without departing from the scope of the invention or any embodiment thereof. Having now described the present invention in detail, the same will be more clearly understood by reference to the following Examples, which are included herewith for purposes of illustration only and are not intended to be limiting of the invention.

### EXAMPLES

### Small-Molecule Screening Assays

Screens for the identification of therapeutics useful in the treatment of synovial sarcoma have been reported previously. See, in particular, US Patent Application Publication No. 2014/0288162A1. Similar methods have been utilized in the instant examples to identify specific compounds having utility in the treatment of synovial sarcoma.

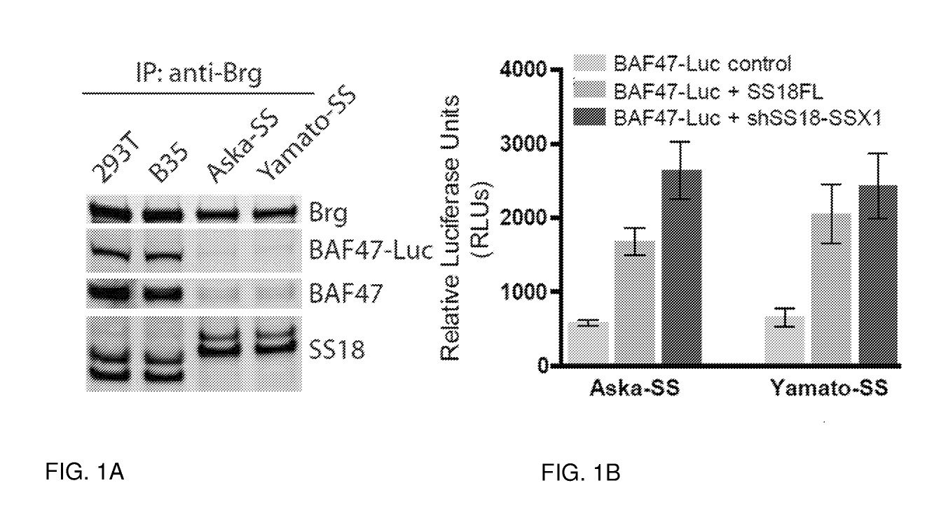

A BAF47-Luciferase gain-of-function screening approach in an Aska-SS synovial sarcoma cell line was used to screen a library of small-molecule compounds. As shown in FIGS. 1A-1B, Luciferase-tagged BAF47 is unstable in SS cells. See also US Patent Application Publication No. 2014/0288162A1. Luciferase-tagged BAF47 activity is induced, however, following reformation of WT BAF complexes. Specifically, the cell lines indicated in FIG. 1A (293T, B35, Aska-SS, and Yamato-SS) were stably infected using a lentiviral construct expressing C-terminally luciferase tagged human BAF47 (also known as SMARCB1, hSNF5, and INI1). Cells were selected with blastocydin following infection to achieve a pure population. Selected cells were harvested, and total nuclear protein was extracted, 20 μg of which was run on by SDS-PAGE for immunoblotting analysis for BAF47 (where both BAF47Luc and endogenous BAF47 are recognized), BRG1, and SS18 (where both SS18 WT and the SS18-SSX oncogenic fusion are recognized). Immunoprecipitation of BAF complexes in WT (293T, B35) versus SS (Aska, Yamato) cells modified with BAF47-Luc is shown in FIG. 1A.

BAF47-Luciferase modified Aska-SS synovial sarcoma cells were co-infected with either inducible shSS18-SSX or inducible SS18WT, and relative luminescence intensity was measured using a luminometer as compared to control cells co-infected with an empty vector. As shown in FIG. 1B, both experimental conditions for promoting reformation of the BAF complex achieve >2.5 fold increase in luciferase activity, reflecting increased cellular stability of BAF47.

A total of 97,489 compounds from the Broad Institute DOS (Diversity Oriented Synthesis) library (see, e.g., Comer et al. (2011) Proc. Nat'l Acad. Sci. USA 108:6751; Lowe et al. (2012) J. Org. Chem. 77:7187; Marcaurelle et al. (2010) J. Am. Chem. Soc. 132:16962) were screened for activity using the cell lines described above. Briefly, BAF47-Luc cells were expanded and validated for performance (as in FIGS. 1A-1B) prior to the screening assays. Cell lines were then then shipped to the screening facility for expansion, re-validation, and full high-throughput screening. Assay format: 1536 well plates, 48 hour incubation with 16 uM compound, in duplicate. Data were collected and analyzed using Spotfire software.

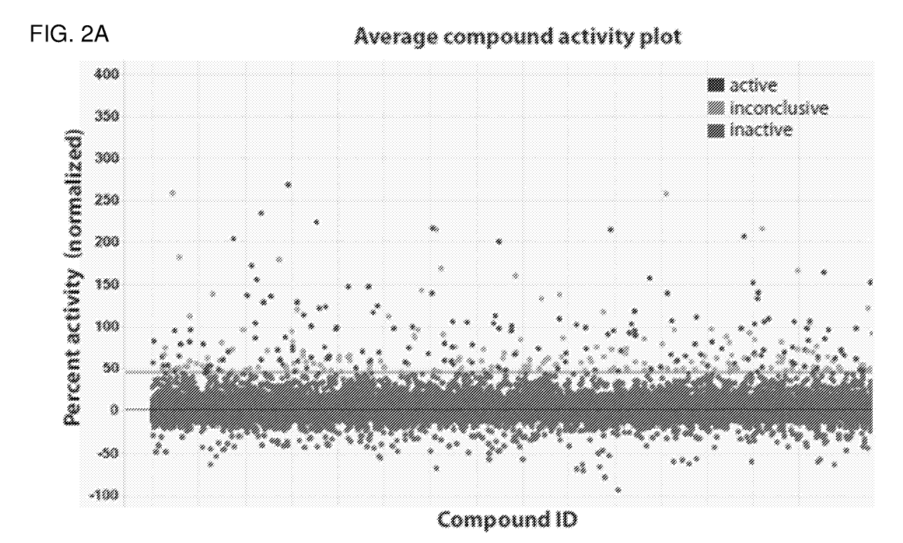

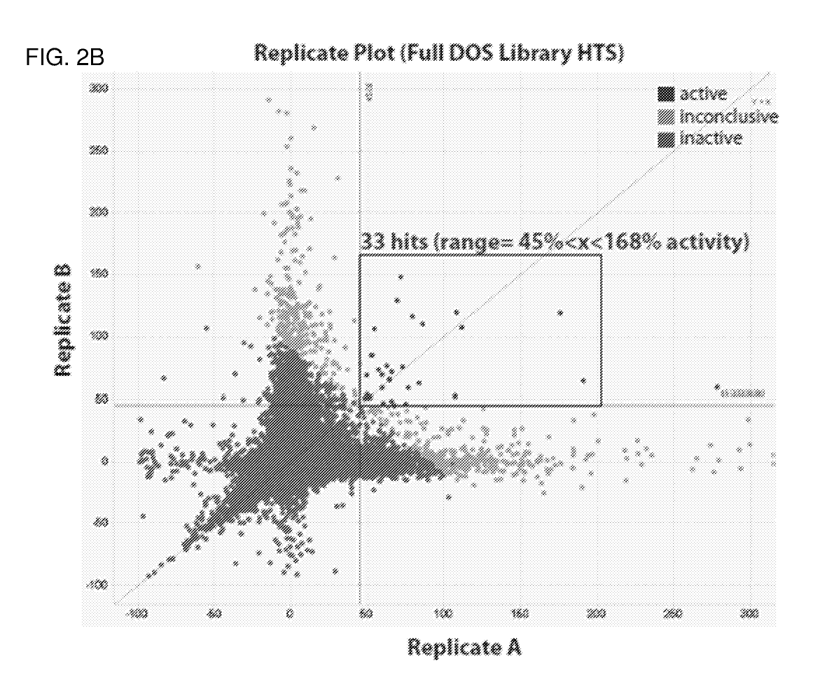

FIG. 2A illustrates a compound activity plot showing the activity of positive hit compounds, inconclusive compounds, and inactive compounds. Active compounds scored at >45% activity (3×SD of mean), with values normalized to the DMSO-treated population (no positive control). FIG. 2B shows a replicate plot, which reveals 33 positive hits with >45% activity (luminescence value); 0.03% hit rate from total library of 97,489 DOS compounds screened in 4 parallel HTS runs. A total of 591 inconclusive compounds were identified (0.6%), which showed signal in only one of two replicates. Cell/assay performance was validated by repeating validation plates at the end of the final run.

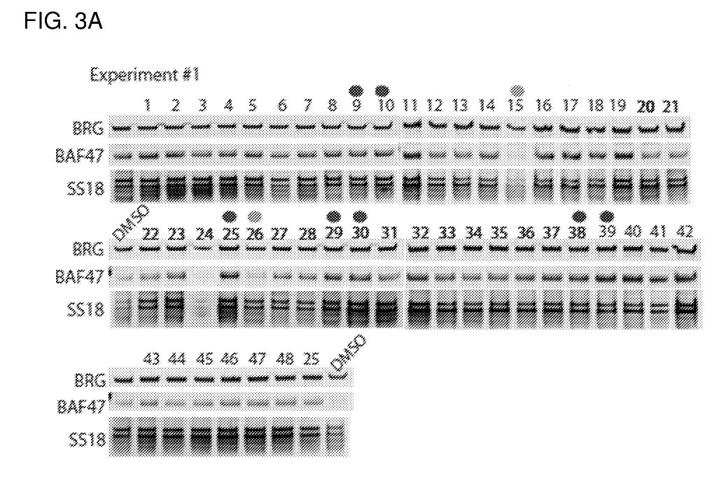

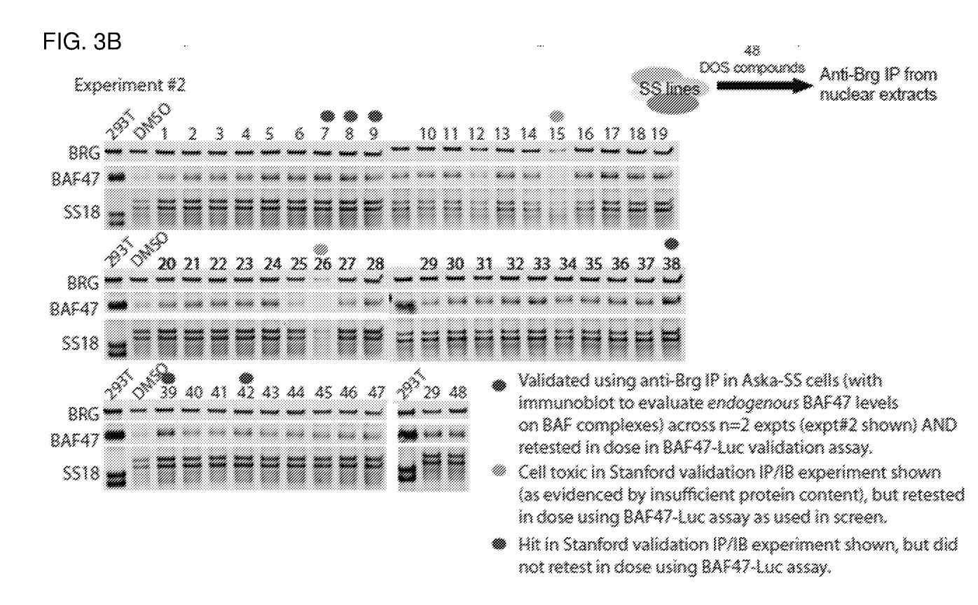

As illustrated in FIGS. 3A-3B, 48 compounds (33 positive and 15 borderline) from the BAF47-Luciferase screens were further validated using unmodified human Aska-SS cells and endogenous protein levels. Importantly, these validations were performed using naïve SS cells (Aska-SS cells), not modified by any lentivirus, and at low passage.

Cells were plated at 300,000 cells per well in 6-well plates and incubated with 16 uM of each of the test compounds (in DMSO) for 48 hours (screening time point). Cells were harvested with RIPA buffer (100 ul) and allowed to incubate with rotation for 30 minutes prior to centrifugation and protein quantification. 70 μg total protein was used for anti-Brg immunoprecipitation using 1.25 μg antibody in total volume of 200 μl IP buffer/RIPA lysate mixture. Immunoblots were performed for control and compound-treated cells, using antibodies to BRG, SS18, and BAF47. Levels of each protein are reflected on the blot and an accompanying Excel file. Endogenous complexes were isolated from these cells, and the BAF47 levels of complexes were assessed via anti-BRG1 immunoprecipitation. The blots shown from Experiment 1 (FIG. 3A) were not normalized for total protein input, whereas the blots shown from Experiment 2 (FIG. 3B) were normalized to a total of 70 μg input protein.

As shown in this experiment, selected compounds increased BAF47 levels and hits were called as those with >3.5 fold increase in the BAF47/Brg ratio relative to DMSO treated cells.

The following exemplary active compounds were identified in the above screening assays:

| Pubchem CID | Structure |
|---|---|
| 44507118 | 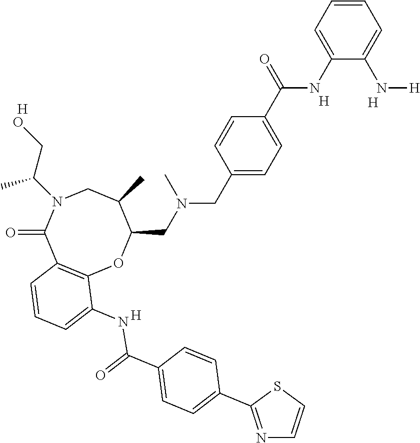 |
| 44507997 | 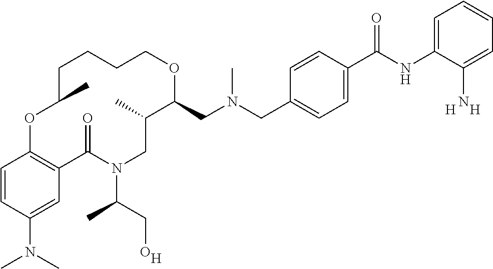 |
| 44495344 | 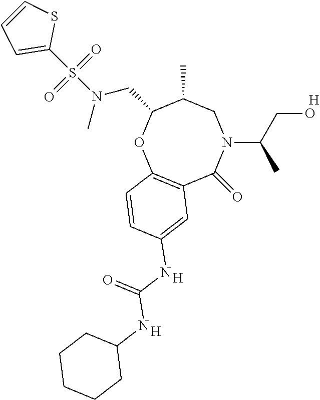 |
| 60194069 | 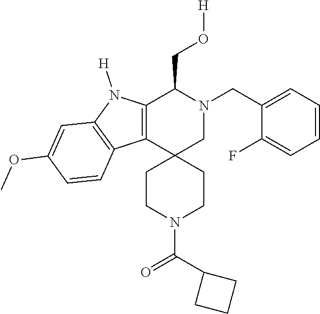 [SMILES: COc1ccc2c3c([nH]c2c1)[C@@H](CO)N(Cc1ccccc1F)CC31CCN(C(=O)C2CCC2)CC1] |
| 60194068 | 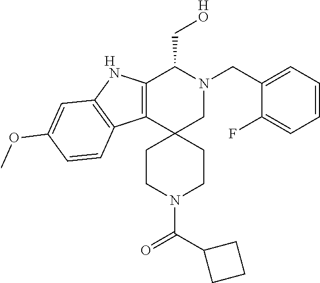 |

All patents, patent publications, and other published references mentioned herein are hereby incorporated by reference in their entireties as if each had been individually and specifically incorporated by reference herein.

While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined by reference to the appended claims, along with their full scope of equivalents.

What is claimed is:

1. A method of treatment, comprising administering to a subject in need thereof a concentration of a compound sufficient to treat synovial sarcoma in the subject, wherein the compound is represented by structural formula (I): 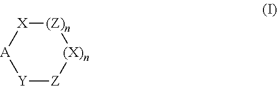
or a stereoisomer, tautomer, or pharmaceutically acceptable salt thereof;
wherein A is an optionally substituted bivalent aryl or heteroaryl group;
each X is independently O or S;
Y is —C(O)NR₁— or —C(S)NR₁—, wherein R₁ is hydrogen, alkyl, alkenyl, alkynyl, aryl, aralkyl, heteroaryl, heteroaralkyl, cycloalkyl, cycloalkenyl, heterocyclyl, or heterocyclylalky, and is optionally substituted with alkyl, alkenyl, alkynyl, alkoxy, alkanoyl, alkylamino, alkylthio, aryl, aryloxy, arylamino, aralkyl, aralkoxy, aralkanoyl, aralkamino, heteroaryl, heteroaryloxy, heteroarylamino, heteroaralkyl, heteroaralkoxy, heteroaralkanoyl, heteroaralkamino, cycloalkyl, cycloalkenyl, cycloalkoxy, cycloalkanoyl, cycloalkamino, heterocyclyl, heterocyclyloxy, heterocyclylamino, heterocyclylalky, heterocyclylalkoxy, heterocyclylalkanoyl, heterocyclylalkamino, hydroxyl, thio, amino, amido, alkanoylamino, aroylamino, aralkanoylamino, alkylcarboxy, alkylcarbonyl, aminocarbonyl, alkylaminocarbonyl, carboxy, carbonate, carbamate, guanidinyl, urea, halo, trihalomethyl, cyano, nitro, phosphoryl, sulfonyl, sulfonamido, or azido;
each Z is independently an optionally substituted C₂₋₆ alkylene group; and
each n is independently either 0 or 1.

2. The method of claim 1, wherein in the compound of structural formula (I):
A is 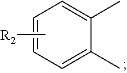
R₂ is hydrogen, —N(R₃)₂, —NR₃C(O)R₄, or —NR₃C(O)N(R₄)₂;
each R₃ is independently hydrogen or an optionally substituted alkyl group; and
each R₄ is independently hydrogen, hydrogen, alkyl, alkenyl, alkynyl, aryl, aralkyl, heteroaryl, heteroaralkyl, cycloalkyl, cycloalkenyl, heterocyclyl, or heterocyclylalky, and is optionally substituted with alkyl, alkenyl, alkynyl, alkoxy, alkanoyl, alkylamino, alkylthio, aryl, aryloxy, arylamino, aralkyl, aralkoxy, aralkanoyl, aralkamino, heteroaryl, heteroaryloxy, heteroarylamino, heteroaralkyl, heteroaralkoxy, heteroaralkanoyl, heteroaralkamino, cycloalkyl, cycloalkenyl, cycloalkoxy, cycloalkanoyl, cycloalkamino, heterocyclyl, heterocyclyloxy, heterocyclylamino, heterocyclylalky, heterocyclylalkoxy, heterocyclylalkanoyl, heterocyclylalkamino, hydroxyl, thio, amino, amido, alkanoylamino, aroylamino, aralkanoylamino, alkylcarboxy, alkylcarbonyl, aminocarbonyl, alkylaminocarbonyl, carboxy, carbonate, carbamate, guanidinyl, urea, halo, trihalomethyl, cyano, nitro, phosphoryl, sulfonyl, sulfonamido, or azido.

3. The method of claim 2, wherein A is

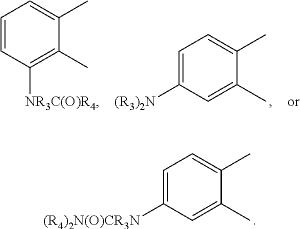

4. The method of claim 3, wherein A is

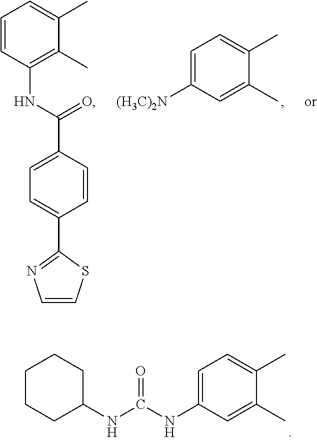

5. The method of claim 1, wherein in the compound of structural formula (I):
X is O.

6. The method of claim 1, wherein in the compound of structural formula (I):
Y is —C(O)NR₁—.

7. The method of claim 1, wherein in the compound of structural formula (I):
each Z is an optionally substituted C₃ alkylene group.

8. The method of claim 1, wherein in the compound of structural formula (I):
n is 1.

9. The method of claim 8, wherein the compound is represented by structural formula (IA):

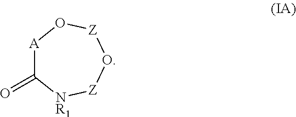

10. The method of claim 9, wherein the compound is represented by structural formula (IA1): 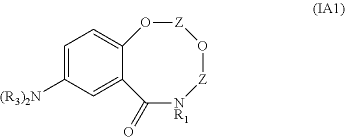
wherein each R₃ is independently hydrogen or an optionally substituted alkyl group.

11. The method of claim 10, wherein the compound is represented by structural formula (IA2): 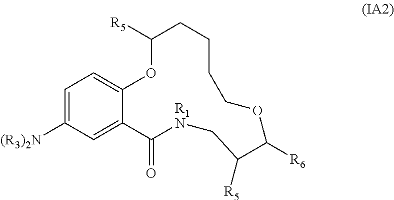
wherein each R₅ is independently an optionally substituted alkyl group; and
R₆ is alkyl, alkenyl, alkynyl, aminoalkyl, aminoalkenyl, aminoalkynyl, aryl, aralkyl, heteroaryl, heteroaralkyl, cycloalkyl, cycloalkenyl, heterocyclyl, or heterocyclylalky, and is optionally substituted with alkyl, alkenyl, alkynyl, alkoxy, alkanoyl, alkylamino, alkylthio, aryl, aryloxy, arylamino, aralkyl, aralkoxy, aralkanoyl, aralkamino, heteroaryl, heteroaryloxy, heteroarylamino, heteroaralkyl, heteroaralkoxy, heteroaralkanoyl, heteroaralkamino, cycloalkyl, cycloalkenyl, cycloalkoxy, cycloalkanoyl, cycloalkamino, heterocyclyl, heterocyclyloxy, heterocyclylamino, heterocyclylalky, heterocyclylalkoxy, heterocyclylalkanoyl, heterocyclylalkamino, hydroxyl, thio, amino, amido, alkanoylamino, aroylamino, aralkanoylamino, alkylcarboxy, alkylcarbonyl, aminocarbonyl, alkylaminocarbonyl, carboxy, carbonate, carbamate, guanidinyl, urea, halo, trihalomethyl, cyano, nitro, phosphoryl, sulfonyl, sulfonamido, or azido.

12. The method of claim 11, wherein in the compound of structural formula (IA2):
R₆ is an aminoalkyl group substituted with an arylalkyl group that is optionally substituted with an aryl-substituted amido group.

13. The method of claim 1, wherein in the compound of structural formula (I):
n is 0.

14. The method of claim 13, wherein the compound is represented by structural formula (IB):

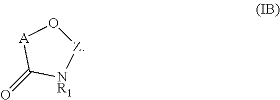

15. The method of claim 14, wherein the compound is represented by structural formula (IB1): 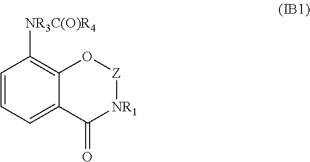
wherein each R₃ is independently hydrogen or an optionally substituted alkyl group; and
each R₄ is independently hydrogen, hydrogen, alkyl, alkenyl, alkynyl, aryl, aralkyl, heteroaryl, heteroaralkyl, cycloalkyl, cycloalkenyl, heterocyclyl, or heterocyclylalky, and is optionally substituted with alkyl, alkenyl, alkynyl, alkoxy, alkanoyl, alkylamino, alkylthio, aryl, aryloxy, arylamino, aralkyl, aralkoxy, aralkanoyl, aralkamino, heteroaryl, heteroaryloxy, heteroarylamino, heteroaralkyl, heteroaralkoxy, heteroaralkanoyl, heteroaralkamino, cycloalkyl, cycloalkenyl, cycloalkoxy, cycloalkanoyl, cycloalkamino, heterocyclyl, heterocyclyloxy, heterocyclylamino, heterocyclylalky, heterocyclylalkoxy, heterocyclylalkanoyl, heterocyclylalkamino, hydroxyl, thio, amino, amido, alkanoylamino, aroylamino, aralkanoylamino, alkylcarboxy, alkylcarbonyl, aminocarbonyl, alkylaminocarbonyl, carboxy, carbonate, carbamate, guanidinyl, urea, halo, trihalomethyl, cyano, nitro, phosphoryl, sulfonyl, sulfonamido, or azido.

16. The method of claim 15, wherein the compound is represented by structural formula (IB2): 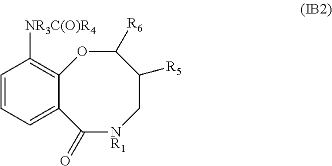
wherein R₅ is an optionally substituted alkyl group; and
R₆ is alkyl, alkenyl, alkynyl, aminoalkyl, aminoalkenyl, aminoalkynyl, aryl, aralkyl, heteroaryl, heteroaralkyl, cycloalkyl, cycloalkenyl, heterocyclyl, or heterocyclylalky, and is optionally substituted with alkyl, alkenyl, alkynyl, alkoxy, alkanoyl, alkylamino, alkylthio, aryl, aryloxy, arylamino, aralkyl, aralkoxy, aralkanoyl, aralkamino, heteroaryl, heteroaryloxy, heteroarylamino, heteroaralkyl, heteroaralkoxy, heteroaralkanoyl, heteroaralkamino, cycloalkyl, cycloalkenyl, cycloalkoxy, cycloalkanoyl, cycloalkamino, heterocyclyl, heterocyclyloxy, heterocyclylamino, heterocyclylalky, heterocyclylalkoxy, heterocyclylalkanoyl, heterocyclylalkamino, hydroxyl, thio, amino, amido, alkanoylamino, aroylamino, aralkanoylamino, alkylcarboxy, alkylcarbonyl, aminocarbonyl, alkylaminocarbonyl, carboxy, carbonate, carbamate, guanidinyl, urea, halo, trihalomethyl, cyano, nitro, phosphoryl, sulfonyl, sulfonamido, or azido.

17. The method of claim 16, wherein in the compound of structural formula (IB2):
R₆ is an aminoalkyl group substituted with an arylalkyl group that is optionally substituted with an aryl-substituted amido group.

18. The method of claim 14, wherein the compound is represented by structural formula (IC1): 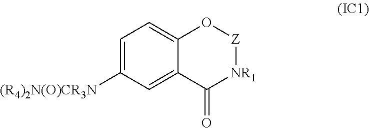
wherein each R₃ is independently hydrogen or an optionally substituted alkyl group; and
each R₄ is independently hydrogen, hydrogen, alkyl, alkenyl, alkynyl, aryl, aralkyl, heteroaryl, heteroaralkyl, cycloalkyl, cycloalkenyl, heterocyclyl, or heterocyclylalky, and is optionally substituted with alkyl, alkenyl, alkynyl, alkoxy, alkanoyl, alkylamino, alkylthio, aryl, aryloxy, arylamino, aralkyl, aralkoxy, aralkanoyl, aralkamino, heteroaryl, heteroaryloxy, heteroarylamino, heteroaralkyl, heteroaralkoxy, heteroaralkanoyl, heteroaralkamino, cycloalkyl, cycloalkenyl, cycloalkoxy, cycloalkanoyl, cycloalkamino, heterocyclyl, heterocyclyloxy, heterocyclylamino, heterocyclylalky, heterocyclylalkoxy, heterocyclylalkanoyl, heterocyclylalkamino, hydroxyl, thio, amino, amido, alkanoylamino, aroylamino, aralkanoylamino, alkylcarboxy, alkylcarbonyl, aminocarbonyl, alkylaminocarbonyl, carboxy, carbonate, carbamate, guanidinyl, urea, halo, trihalomethyl, cyano, nitro, phosphoryl, sulfonyl, sulfonamido, or azido.

19. The method of claim 18, wherein the compound is represented by structural formula (IC2): 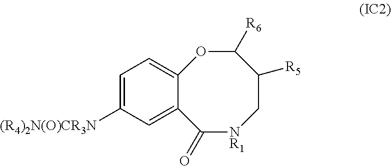
wherein R₅ is an optionally substituted alkyl group; and
R₆ is alkyl, alkenyl, alkynyl, aminoalkyl, aminoalkenyl, aminoalkynyl, aryl, aralkyl, heteroaryl, heteroaralkyl, cycloalkyl, cycloalkenyl, heterocyclyl, or heterocyclylalky, and is optionally substituted with alkyl, alkenyl, alkynyl, alkoxy, alkanoyl, alkylamino, alkylthio, aryl, aryloxy, arylamino, aralkyl, aralkoxy, aralkanoyl, aralkamino, heteroaryl, heteroaryloxy, heteroarylamino, heteroaralkyl, heteroaralkoxy, heteroaralkanoyl, heteroaralkamino, cycloalkyl, cycloalkenyl, cycloalkoxy, cycloalkanoyl, cycloalkamino, heterocyclyl, heterocyclyloxy, heterocyclylamino, heterocyclylalky, heterocyclylalkoxy, heterocyclylalkanoyl, heterocyclylalkamino, hydroxyl, thio, amino, amido, alkanoylamino, aroylamino, aralkanoylamino, alkylcarboxy, alkylcarbonyl, aminocarbonyl, alkylaminocarbonyl, carboxy, carbonate, carbamate, guanidinyl, urea, halo, trihalomethyl, cyano, nitro, phosphoryl, sulfonyl, sulfonamido, or azido.

20. The method of claim 19, wherein in the compound of structural formula (IC2):
R₆ is an alkyl group substituted with a sulfonamide group that is optionally substituted with a heteroaryl group.

21. A method of treatment, comprising administering to a subject in need thereof a concentration of a compound sufficient to treat synovial sarcoma in the subject, wherein the compound is represented by structural formula (II): 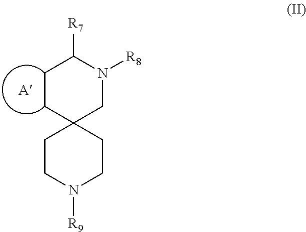
or a stereoisomer, tautomer, or pharmaceutically acceptable salt thereof;
wherein A′ is an optionally substituted aryl or heteroaryl ring system; and
R₇, R₈, and R₉ are each independently, alkyl, alkenyl, alkynyl, acyl, aryl, aralkyl, heteroaryl, heteroaralkyl, cycloalkyl, cycloalkenyl, heterocyclyl, or heterocyclylalky, and is optionally substituted with alkyl, alkenyl, alkynyl, alkoxy, alkanoyl, alkylamino, alkylthio, aryl, aryloxy, arylamino, aralkyl, aralkoxy, aralkanoyl, aralkamino, heteroaryl, heteroaryloxy, heteroarylamino, heteroaralkyl, heteroaralkoxy, heteroaralkanoyl, heteroaralkamino, cycloalkyl, cycloalkenyl, cycloalkoxy, cycloalkanoyl, cycloalkamino, heterocyclyl, heterocyclyloxy, heterocyclylamino, heterocyclylalky, heterocyclylalkoxy, heterocyclylalkanoyl, heterocyclylalkamino, hydroxyl, thio, amino, amido, alkanoylamino, aroylamino, aralkanoylamino, alkylcarboxy, alkylcarbonyl, aminocarbonyl, alkylaminocarbonyl, carboxy, carbonate, carbamate, guanidinyl, urea, halo, trihalomethyl, cyano, nitro, phosphoryl, sulfonyl, sulfonamido, or azido.

22. The method of claim 21, wherein in the compound of structural formula (II):
A′ is an optionally substituted bicyclic aryl or heteroaryl ring system.

23. The method of claim 21, wherein in the compound of structural formula (II):
R₇ is an alkyl group optionally substituted with hydroxyl, thio, amino, carboxy, carbonate, carbamate, guanidinyl, urea, halo, trihalomethyl, cyano, nitro, phosphoryl, sulfonyl, sulfonamido, or azido.

24. The method of claim 21, wherein in the compound of structural formula (II):
R₈ is an aryl, aralkyl, heteroaryl, heteroaralkyl group optionally substituted with hydroxyl, thio, amino, carboxy, carbonate, carbamate, guanidinyl, urea, halo, trihalomethyl, cyano, nitro, phosphoryl, sulfonyl, sulfonamido, or azido.

25. The method of claim 21, wherein in the compound of structural formula (II):
R₉ is an acyl group optionally substituted with hydroxyl, thio, amino, carboxy, carbonate, carbamate, guanidinyl, urea, halo, trihalomethyl, cyano, nitro, phosphoryl, sulfonyl, sulfonamido, or azido.

26. The method of claim 21, wherein the compound is represented by structural formula (IIA): 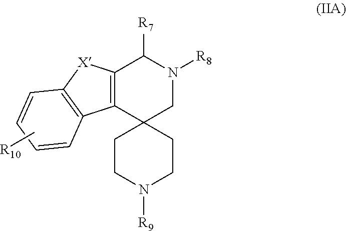
wherein X′ is N—R₁₁, O, or S;
R₁₀ is an alkoxy, alkanoyl, alkylamino, or alkylthio group; and
R₁₁ is hydrogen or alkyl.

27. The method of claim 26, wherein in the compound of structural formula (IIA):
R₇ is an alkyl group optionally substituted with hydroxyl, thio, amino, carboxy, carbonate, carbamate, guanidinyl, urea, halo, trihalomethyl, cyano, nitro, phosphoryl, sulfonyl, sulfonamido, or azido;
R₈ is an aryl, aralkyl, heteroaryl, heteroaralkyl group optionally substituted with hydroxyl, thio, amino, carboxy, carbonate, carbamate, guanidinyl, urea, halo, trihalomethyl, cyano, nitro, phosphoryl, sulfonyl, sulfonamido, or azido; and
R₉ is an acyl group optionally substituted with hydroxyl, thio, amino, carboxy, carbonate, carbamate, guanidinyl, urea, halo, trihalomethyl, cyano, nitro, phosphoryl, sulfonyl, sulfonamido, or azido.

28. The method of any one of claims 1-27, wherein the treatment modulates stability of a BAF complex in the subject.

29. The method of claim 28, wherein the treatment stabilizes formation of a normal BAF complex in the subject.
